(19) United States
(12) Patent Application Publication
(10) Pub. No.: US 2024/0401115 A1
(43) Pub. Date: Dec. 5, 2024

(54) METHOD AND DEVICE FOR DESALTING AND CONCENTRATING OR ANALYSING A SAMPLE OF NUCLEIC ACIDS

(71) Applicant: ADELIS, Labege (FR)

(72) Inventors: Frederic GINOT, Labege (FR); Audrey BOUTONNET, Labege (FR)

(21) Appl. No.: 18/697,742

(22) PCT Filed: Oct. 13, 2022
(86) PCT No.: PCT/EP2022/078580

§ 371 Date: Apr. 2, 2024

(30) Foreign Application Priority Data
Oct. 14, 2021 (FR) FR2110921

(51) Int. Cl. C12Q 1/6825; B01L 3/00; C12Q 1/6806

(52) U.S. Cl. CPC C12Q 1/6825; B01L 3/502761; C12Q 1/6806; B01L 2200/0647; B01L 2300/0654; B01L 2400/0406; B01L 2400/0421

(57) ABSTRACT

The method for desalting and concentrating a nucleic acid sample more conductive than an analysis buffer comprises at least one iteration of an alternation of:
a step of the laminar flow of the sample through a capillary in a first direction of flow, the capillary being equipped with a local restriction in its cross-section and comprising the analysis buffer, during which the sample is subjected to a first difference in electric potential whose action on the nucleic acid molecules is opposite to the direction of flow and causes the nucleic acid molecules to be retained in the capillary;
a step of the laminar flow of the sample, in a second direction of flow opposite to the first direction of flow.

### TECHNICAL FIELD OF THE INVENTION

The present invention relates to a method and a device for desalting and concentrating or analysing a nucleic acid sample. It applies, in particular, to the concentration and analysis of circulating DNA fragments, and to medical diagnosis, in particular for certain cancers.

### STATE OF THE ART

The circulating DNA in blood plasma is currently the focus of intense clinical research in oncology, since it can contain the genomic DNA mutations present in tumour cells, which directs the therapy to be administered to the patient. These mutations, or other genomic changes, can be used as biomarkers to monitor the progress of the cancer. However, in general, there are no easy-to-monitor biomarkers in the context of cancers.

For example, for pancreatic cancer the only monitoring biomarker used routinely at the moment is Ca 19.9 (carbohydrate antigen 19.9) measured in the circulating blood. This biomarker has a sensitivity of 79% and specificity of 82% in this context. However, the Ca 19.9 level is high in other, benign, pancreatic pathologies such as acute pancreatitis, and in other digestive cancers. Research for innovative biomarkers is therefore necessary, not only to improve the early diagnosis of pancreatic cancer, but also to evaluate the prognosis for these patients, their monitoring and their response to treatments.

It has been clearly established that blood carries a small amount of free circulating DNA coming from the release of genetic material by the tissues. This circulating cell-free DNA (cfDNA) is in the form of double-stranded DNA with an average size of 150-180 bp corresponding to the winding of DNA around the nucleosome. Its lifespan is less than two hours, before it is filtered and eliminated from the bloodstream by the spleen, liver and kidneys. All studies are in agreement on a baseline quantitative detection of cfDNA in healthy individuals compared to different clinical situations such as CerebroVascular Accidents (CVA) and myocardial infarctions, intensive muscular exercises, acute renal failure, hepatic cytolysis, trauma, surgery, cancer, the presence of a foetus during pregnancy, etc.

In the case of cancer, recent advances in molecular biology and DNA sequencing have made it possible to identify many gene mutations involved in oncogenesis based on circulating tumour DNA (ctDNA). A study in 2014 by Bettegowda et al. showed that the level of circulating tumour DNA could be correlated to the tumour load and the stage of the cancer: it would be lower for localised cancers than for metastatic cancers.

The routine use of ctDNA as a tumour biomarker offers prospects in the screening, diagnosis, therapeutic decision-making and monitoring of cancer patients. Only some ten studies have looked at ctDNA in patients with a resected pancreatic cancer. In most of these publications, the ctDNA is detected by looking for KRAS mutations by PCR (acronym for Polymerase Chain Reaction). But pancreatic adenocarcinoma has many other mutations involving the genes Tp53 or p16. Therefore, clinically it is more advantageous to carry out additional analyses of these genes by next generation sequencing (NGS).

The mechanisms contributing to the production of cfDNA comprise apoptosis, necrosis, NETosis, the active secretion of cells that results in the release of DNA in the form of extracellular vehicles (EV), the alteration of clearing mechanisms. These mechanisms can be distinguished through the size of cfDNA detected in the blood circulation.

Therefore, the physiological and pathological phenomena at the origin of the passive and active release of free DNA are distinguished based on their size.

With regard to pancreatic cancer, some very recent articles discuss the benefit of measuring the size of the cfDNA. Thus, Lapin M. et al. (“Fragment size and level of cell-free DNA provide prognostic information in patients with advanced pancreatic cancer”. J Transl Med, 2018. 16 (1): p. 300.) showed, in 2018, that the size of the ctDNA fragments and the ctDNA concentrations can be used to predict the outcome of the disease in patients with advanced pancreatic cancer. Zvereva M. et al. (“Circulating tumour-derived KRAS mutations in pancreatic cancer cases are predominantly carried by very short fragments of cell-free DNA.” EBioMedicine, 2020. 55: p. 102462.) showed that the proportion of cases with KRAS mutations detectable by PCR is inversely proportional to the increase in the length of amplicons; only half of the cases with a PCR of 218 bp have a detectable ctDNA, compared to those detected with amplicons of less than 80 bp. And lastly; Liu X. et al. (“Enrichment of short mutant cell-free DNA fragments enhanced detection of pancreatic cancer.” EBioMedicine, 2019. 41: p. 345-356.) showed that the detection of mutations can be enriched by creating NGS processes tailored to fragments with very short sizes.

With regard to the current limits in the routine diagnostic use of cfDNA as a biomarker, the main obstacles are linked to:
a/pre-analytical processes; the cfDNA can be contaminated by the artifactual release of leucocyte DNA in vitro, thus it is essential to use standardised protocols.
b/the standardisation of the extraction of cfDNA and its quantification; there is a great variety of “internal protocols”, but in most cases there is no genuine strategy for the quality control and standardisation of qualification and normalisation methods for the cfDNA extracted.
c/the absence of a general pan-tumour method making it possible to characterise the cfDNA; in the absence of somatic genome abnormalities, the characterisation of the size profile of the cfDNA, and therefore its origin, offers many advantages, but the current approaches are either too complex and costly, or not sufficiently sensitive to profile all patients, whatever the stage of the disease, including healthy individuals.

The device described in documents PCT/FR2016/051774 and WO/2017/009566 enables the size profile of the cfDNA to be determined easily and economically. In order to analyse the DNA, the device operates in two steps, concentration and separation, performed in line. First of all, the DNA is concentrated by means of a capillary system formed of the junction of one capillary and another with a larger cross-section. The researchers make a solution containing the DNA flow in a laminar way in the large capillary, and use an electric field to slow down the migration. Because of the shearing from the laminar flow, this counter-electrophoresis produces a transverse force, dependent on the size of the DNA, which pushes the DNA towards the walls. The change in the speed of flow and in the electric field at the location of the constriction makes it possible to halt the DNA and concentrate it like a “clump”. In effect, upstream from the constriction, the flow and counter-electrophoresis are slow, causing a weak transverse force. The DNA is therefore in the bulk of the flow, and moves towards the constriction. On the other hand, downstream from the constriction, the flow and counter-electrophoresis are rapid, pressing the DNA firmly against the wall at a location where the laminar flow is very low and where the counter-electrophoresis dominates. The DNA therefore moves back towards the constriction, by counter-electrophoresis, along the wall. This clump is then released by the progressive decrease in the electric field, which also makes it possible to carry out the separation operation according to the size of the fragments. This technology is called “uLAS” in the rest of the description.

FIG. 1 is a schematic diagram of such a device 40. This device 40 can be installed in an Agilent (registered trademark) CE (acronym for “Capillary electrophoresis”) in the place of a standard capillary.

The total length of the device is approximately 30 cm, the minimum length that can be installed in an Agilent CE, which makes it possible to apply pressure and voltage to the terminals of the capillary device. It comprises, from the inlet 41 to the outlet 42:
an injection port 43, 5.5 cm long and 40 μm in diameter; this port serves as a filter, and makes it possible to position the injection chamber inside the Agilent CE's ventilated cartridge, beyond the electrode;
an injection chamber 44, formed of a two cm long capillary segment, which has a large inner diameter, for example 350 μm;
a separation capillary 45, with an inner diameter of 40 μm and nine cm long. The measurement 46 of the fluorescence of DNA molecules takes place seven cm after the concentration junction;
a distal capillary 47, with an inner diameter of 100 μm and 13 cm long, which makes it possible to join the outlet vial onto the Agilent CE. This capillary 47 has a larger diameter than the capillary 45 so that its electrical resistance and hydraulic resistance are small compared to the combined resistances of the injection port 43 and separation capillary 45. If the separation capillary 45 were to go right up to the outlet 42, the voltage and pressure to be applied to retain small DNA fragments would be greater than what the instrument permits.

The dimensions given above are only examples. For example, a slightly greater capillary dimension can be adapted to very large DNA fragments (200 kb), or the method of assembling the capillaries can be modified to carry out DNA splitting, for example to boost the amount of DNA that can be stored at a junction, at the expense of the analysis resolution.

The concentration area is located at the junction between the injection chamber 44 and the separation chamber 45. In the rest of the description, the distal capillary 47 is considered to play no role in the concentration of DNA fragments.

FIG. 2 shows the typical sequence of a DNA analysis with this device 40. The distal capillary 47 is not shown in this figure.

During an operation 51, the sample is injected into the device, by applying pressure for a given duration.

During an operation 52, the injected sample is pushed by pressure to the centre of the injection chamber 44.

During operations 53 and 54, the DNA is concentrated at the concentration junction 48 of the injection chamber 44, by applying a pressure and a voltage. At the start of the concentration, the separation capillary 45 fills with the sample solution, except for the largest DNA fragments, which remain retained at the concentration junction 48.

During an operation 55, the DNA retained at the concentration junction 48 is separated according to size by a progressive decrease in the electric field applied between the inlet 41 and the outlet 42, the pressure generally being maintained constant. The DNA fragments are detected by fluorescence during their passage in front of the detector 46.

Typically, during the concentration step the following physical values are applied to retain DNA fragments as small as 100 bp:
voltage: 25,000 V
pressure: 10 bars
the solution's viscosity and resistivity in the capillary are respectively 40 mPa·s and 12.4 Ω.m at 25° C.

In the separation capillary 45, the following are obtained:
an electric field of 1500 V/cm; and
an average fluid speed of 9 mm/s.

The value of the electric field given above is valid as long as the conductivity of the sample is close to, or less than, the conductivity of the analysis buffer (about 12.4 Ω.m at 25° C.). This is generally the case when the DNA is purified beforehand, and taken up in a buffer with low conductivity.

But when the sample contains salts, the electrical resistance of the separation capillary drops suddenly at the start of the concentration (step 53), and the electric field at the location of the concentration junction 48 drops abruptly, and would no longer be sufficient to concentrate the smallest DNA fragments.

The phenomenon is described analytically and schematically below, for a device that would be fully symmetrical on each side of the injection chamber 44.

When a voltage U is applied to the terminals of the capillary device, an electrical current is established, according to Ohm's law U=Ri, where R is the electrical resistance of the device, and i the electric current.

Locally, at all points along the device, Ohm's law is expressed by

$$E = \frac{ρ}{s} i ,$$

where E is the electric field in Volts per metre, p the electrical resistivity of the electrolyte in Ohm metres, and S the cross-section of the capillary device in square metres, at the point considered, with i, expressed in amperes, being constant over the entire length of the device.

When a sample with salts, therefore with a low resistivity p, is injected into a device, and then pressure and voltage are applied to concentrate it, after a short time, the port 43 of the device is filled with the analysis buffer, typically of the order of p=10 Ω.m, while the separation capillary 45 is filled with the sample solution, typically p=0.7 Ω.m for human plasma, which corresponds to approximately 130 mM NaCl. Because, in the port 43 as in the separation capillary,

$$E = \frac{ρ}{s} i , \qquad [ Math 1 ]$$

the electric field E₁ and E₂ present respectively in the plot and the separation capillary nave the ratio

$$\frac{{E}_{2}}{{E}_{1}} = \frac{{ρ}_{2}}{{ρ}_{1}} = \frac{0.7}{10} = 0.07 . \qquad [ Math 2 ]$$

The electric field in the separation capillary 45, which must retain the DNA in the injection chamber 44, is therefore only 7% of the value of the electric field in the port of the device. To obtain an electric field of 1500 V/cm in the separation capillary, an electric field of 21,400 V/cm would be necessary in the port 43, which cannot be achieved in practice because of the heating by Joule effect that this would generate, in its turn this heating would cause the degassing of the electrolyte in the capillary and the formation of air bubbles cutting the electrical continuity of the electrolyte, reducing to almost zero any electric field downstream, in the separation capillary.

The electric field at the outlet of the concentration junction 48 and at the outlet of the device can be modelled throughout a concentration/separation method as described in the steps 53 to 55, for a device 40 to which 25,000 volts are applied.

FIG. 3 shows the electric field 70 calculated at one millimetre downstream from the concentration junction 48, as a function of time. At t=0, the sample is in the centre of the injection chamber 44, with a volume equal to half the volume of the injection chamber 44, i.e. approximately 1 μL. The sample has a conductivity of 0.7 Ω.m. The electric field is calculated one mm after the concentration junction 48, not taking into account the effect of the heating by Joule effect of the capillary 45 on the conductivity and viscosity of the solution, nor the electro-osmosis phenomenon that accelerates the flow. The flow rate is therefore 0.68 μL/min throughout the analysis.

During a phase 71, the sample is completely inside the injection chamber 44; the separation capillary and the inlet capillary, as well as the distal capillary, are completely filled with the analysis buffer, which has a conductivity of 12.4 Ω.m. The electric field is 1500 V/cm, a field broadly sufficient to retain the fragments 15 of 100 bp.

During a phase 72, the low-resistivity sample rapidly fills the separation capillary 45, with a volume of 0.11 μL. When the interface between the sample and the analysis buffer arrives at the observation point, 1 mm downstream from the concentration junction, the electric field drops abruptly to approximately 90 V/cm; then rises slightly as the separation capillary fills with sample.

During a phase 73, the sample still partly fills the injection chamber 44, it completely fills the separation capillary 45, and it progressively fills the distal capillary 47, with a volume of 1.02 μL. The electric field slowly rises to 215 V/cm. Such a field is totally insufficient to retain the smallest DNA fragments.

During a phase 74, the sample is completely evacuated from the injection chamber 44, and retreats rapidly from the separation capillary 45, then more slowly from the distal capillary 47; the field increases sharply when the sample no longer fills the observation point, but still fills most of the separation capillary 45.

During a phase 75, the sample with conductivity 0.7 Ω.m is completely evacuated from the device. The initial electric field of 1500 V/cm is again obtained.

At the distal end of the separation capillary 45, before the distal capillary 47, a profile can be seen that is similar, but offset in time since the interface between the sample and the analysis buffer arrives at this location later.

The insufficiency of the electric field during the phases 72 and 73 prevents the concentration of the smallest DNA fragments.

The description with regard to FIGS. 5 to 9 shows experimentally the phenomenon on a calibration sample of DNA, and on an actual sample of circulating DNA. It can be seen that beyond 10 mM NaCl in the sample, the fragments of 100 bp are no longer completely retained, and that beyond 20 mM NaCl, these fragments are practically no longer retained. In addition, FIG. 7, which shows the amperage circulating in the capillary during the concentration step, illustrates the salt removal kinetics of the device.

The following scientific publications are known, which disclose a method of DNA concentration and separation, such a method comprising the application of an electric field combined with a hydrodynamic flow in a capillary comprising a restriction:
Andriamanampisoa et al., “BIABooster: Online DNA Concentration and Size Profiling with a Limit of Detection of 10 fg/μL and Application to High-Sensitivity Characterization of Circulating Cell-Free DNA”, Anal. Chem. 2018, 90, 3766-3774; and
Milon et al., “uLAS technology for DNA isolation coupled to Cas9-assisted targeting for sequencing and assembly of a 30 kb region in plant genome”, Nucleic Acids Research, 2019, Vol. 47, No. 15, 8050-8060.

However, such methods do not make it possible to desalt a nucleic acid sample.

The scientific publication by Davis et al., “Capillary and Microfluidic Gradient Elution Isotachophoresis Coupled to Capillary Zone Electrophoresis for Femtomolar Amino Acid Detection Limits”, Anal. Chem. 2009, 81, 5452-5459, which discloses a method for concentrating and separating amino acid, is also known. However, this method does not make it possible to desalt a nucleic acid sample.

### Presentation of the Invention

The present invention aims to remedy all or part of these drawbacks.

To this end, according to a first aspect, the present invention envisions a method for desalting and concentrating a nucleic acid sample more conductive than an analysis buffer, which method comprises at least one iteration of an alternation of:
a step of the laminar flow of the sample through a capillary in a first direction of flow, the capillary being equipped with at least one local restriction in its cross-section and comprising the analysis buffer, during which the sample is subjected to a first difference in electric potential whose action on the nucleic acid molecules is opposite to the first direction of flow and causes the nucleic acid molecules to be retained in the capillary;
a step of the laminar flow of the sample, in a second direction of flow opposite to the first direction of flow.

In other words:
during the step of the laminar flow of the sample in a capillary equipped with a local restriction in its cross-section, in the first direction of flow, a pressure greater than the pressure in the restriction is applied upstream from the restriction; and
during the step of the laminar flow of the sample in a second direction of flow opposite to the first direction of flow, a pressure lower than the pressure in the restriction is applied upstream from the restriction.

The inventors have discovered that, when the electric field is insufficient to retain small DNA fragments at the concentration junction, these fragments leak into the separation capillary. But the phenomena implemented always push them towards the wall, even if this pressing against the wall is insufficient for the counter-electrophoresis to be stronger than the fluid flow. This results in the migration speed of these DNA fragments being slower than the average speed of the flow. In contrast, the ions forming the salts are too small for the development of a transverse force pushing them towards the wall, and they advance at the average speed of the flow, greater or less than their speed of electrophoresis depending on their positive or negative charge.

Therefore, the concentration operation is stopped before the smallest fragments arrive at the end of the separation capillary 45 and a pressure is applied, on its own or in the presence of a difference in potential, in the opposite direction to the initial pressure to take the volume from the separation capillary back into the injection chamber. Then, a new concentration step is carried out and, possibly, this alternation of return and concentration phases is reiterated. The salts are therefore evacuated progressively from the device, while the DNA molecules are mainly retained.

The small DNA fragments can therefore be analysed correctly, which was impossible with the devices and methods of the prior art.

In some embodiments, during the step of the laminar flow of the sample in the second direction of flow, the sample is subjected to a second difference in electric potential whose action on the nucleic acid molecules is opposite to the first direction of flow.

Note that the second difference in potential can be equal to, greater than or less than the first difference in potential.

Thus, the molecules still close to the walls of the separation capillary return more rapidly into the injection chamber than during a return by pressure alone. In effect, firstly the electrophoresis is added to the flow, and the electrophoresis has a constant speed in a plane transverse to the flow, unlike the fluid laminar flow, which is zero at the wall and maximum at the centre. And, secondly, a transverse force develops, this time pushing the molecules towards the centre of the fluid path (not towards the walls, which is the case in the concentration phase), where the flow is faster.

In some embodiments, the method also comprises a step of measuring the amperage circulating in the capillary during at least one step of flowing in the first direction of flow and a step of selecting a number of iterations of the alternation according to the amperage measured.

Thanks to these provisions, the conductivity of the sample, representative of its salinity, is estimated by means of measuring the current passing through the sample and, based on this salinity, a number of alternations of flows in one direction and then in the other, making it possible to reduce this salinity to an appropriate level, is chosen.

In some embodiments, the number of iterations of the alternation is an increasing function of the amperage of a peak of this measured amperage or of a peak of a derivative of this measured amperage.

The inventors have observed that the height of at least one peak of the measured amperage and the peak of the first derivative of the measured amperage are increasing functions of the salinity of the sample.

In some embodiments, the method also comprises a step of measuring the amperage circulating in the capillary during at least one step of flowing in the first direction of flow and a step of selecting a duration of at least one step of flowing in the first direction of flow according to the amperage measured.

The estimation of the salinity makes it possible to estimate the speed of motion of small nucleic acid fragments. Therefore a flow duration is chosen that prevents these small fragments from exiting from the capillary.

In some embodiments, the duration chosen is a decreasing function of the amperage of a peak of this measured amperage or of a peak of a derivative of this measured amperage.

In some embodiments, the method comprises after an iteration of the alternation, a step of changing the analysis buffer, the new analysis buffer having a lower pH than the analysis buffer previously used.

For example, in a biological sample comprising proteins, some of these proteins are positively charged and therefore attach themselves to the walls of the capillary, and also to the nucleic acids, which are negatively charged. This results in nucleic acids sticking to the walls, which adhesion is detrimental to the analysis. By choosing a buffer with a high pH, most of the proteins are negatively charged, and the sticking of nucleic acids to the walls is reduced, even disappears. But, with a high pH, a separation of the nucleic acids is not as decisive as with a neutral or slightly acidic pH. With a change of buffer, the capacity to separate nucleic acids is restored.

According to a second aspect, the present invention envisions a method for analysing a nucleic acid sample, which comprises the steps of the method for desalting and concentrating a nucleic acid sample that is the subject of the invention and, after the last iteration of the alternation, a step of separation by laminar flow of the sample in the capillary in the first direction of flow, during which the sample is subjected to a difference in electric potential less than or equal to the first difference in potential, whose action on the nucleic acid molecules is opposite to the first direction of flow and causes a partial retention of the nucleic acid molecules in the capillary.

In this way, the nucleic acids are partially retained, which improves their separation according to their size.

In some embodiments, during the separation step the difference in electric potential decreases.

The largest nucleic acid fragments are therefore retained longer in the capillary device, so that the analysis of the sizes of nucleic acids is more fine-grained.

In some embodiments, the analysis method comprises, during or after the separation step, a step of measuring a fluorescence time profile of fluorescent nucleic acid molecules and a step of concentrating into nucleic acid molecules of different lengths, by utilising a fluorescence profile of a calibration sample, in which the concentration is known for each length of fluorescent nucleic acid molecule.

The nucleic acid molecules are made more fluorescent by one of the techniques known to the person skilled in the art, for example by adding an intercalating fluorophore. An intercalating fluorophore is a molecule that fluoresces little in the free state, and fluoresces strongly when it is intercalated between the DNA bases.

An analysis of samples is carried out with offsetting of experimental variations that exist from one day to the next, in terms of the passage time of the DNA in front of the detector and in terms of fluorescence intensity.

According to a third aspect, the present invention envisions a device configured to implement a desalting and concentration method that is the subject of the invention, or an analysis method that is the subject of the invention for analysing a nucleic acid sample more conductive than an analysis buffer, the device comprising:
a capillary equipped with a local restriction in its cross-section and comprising the analysis buffer;
a means for placing the sample in the capillary in laminar flow, in a first direction of flow;
a means for applying a first difference in electric potential during the flow in the first direction, a difference in potential whose action on the nucleic acid molecules is opposite to the first direction of flow and causes the retention of the nucleic acid molecules in the capillary;
a means for placing the sample in the capillary in laminar flow, in a second direction of flow opposite to the first direction of flow; and
a means for controlling the means for placing in laminar flow and the means for applying the first difference in electric potential configured to control at least one iteration of one alternation;
a laminar flow of the sample in the capillary in the first direction of flow, a flow during which the sample is subjected to the first difference in electric potential; and
a laminar flow of the sample in the capillary in the second direction of flow.

In some embodiments, the capillary is formed of a microfluidic channel.

As the particular features, advantages and aims of this analysis method and this device are similar to those of the method for desalting and concentrating or analysing a nucleic acid sample that are the subject of the invention, they are not repeated here.

### BRIEF DESCRIPTION OF THE FIGURES

Other advantages, aims and particular features of the invention will become apparent from the non-limiting description that follows of at least one particular embodiment of the desalting and concentration or analysis method and device that are the subjects of the present invention, with reference to drawings included in an appendix, wherein:

FIG. 1 represents, schematically, a capillary device of the prior art;

FIG. 2 represents a typical sequence of operation of a DNA analysis with the device illustrated in FIG. 1;

FIG. 3 represents an electric field calculated, over time, at one millimetre downstream from the concentration junction of the device illustrated in FIG. 1 operating as illustrated in FIG. 2, when the sample has a conductivity 18 higher than that of the analysis buffer;

FIG. 4 represents an analysis result for a DNA sample without salts, with the device illustrated in FIG. 1 operating as illustrated in FIG. 2;

FIG. 5 represents an analysis result for calibration DNA samples containing more or less salt, with the device illustrated in FIG. 1 operating as illustrated in FIG. 2;

FIG. 6 presents the areas of the peaks of FIG. 5, as a function of the NaCl concentration in the sample;

FIG. 7 shows the amperage circulating in the capillary during the concentration step of the analyses represented in FIG. 5, illustrating the salt removal kinetics of the device illustrated in FIG. 1;

FIG. 8 represents the analysis of a purified circulating DNA sample containing more or less salt, with the device illustrated in FIG. 1 operating as illustrated in FIG. 2;

FIG. 9 represents changes in the area of the first peak and second peak of the graphs in FIG. 8 as a function of the NaCl concentration in the sample, with the device illustrated in FIG. 1 operating as illustrated in FIG. 2;

FIG. 10 represents a typical sequence of operation of a DNA analysis with the method that is the subject of the invention implementing two returns;

FIG. 11 represents an analysis performed with the device illustrated in FIG. 1 operating according to the sequence illustrated in FIG. 10, for samples comprising more or less salt;

FIG. 12 presents the areas of the peaks of FIG. 11, as a function of the NaCl concentration in the sample;

FIG. 13 illustrates the time positioning of two return periods in the timeline of an embodiment of the method, returns during which, because no electric voltage is applied, the electrical current is zero;

FIG. 14 represents the result of an analysis performed with the device illustrated in FIG. 1 operating according to the sequence illustrated in FIG. 10, for previously purified cfDNA samples comprising more or less salt;

FIG. 15 represents changes in the area of the first peak and second peak of the curves in FIG. 14 as a function of the NaCl concentration, with the device illustrated in FIG. 1 operating as illustrated in FIG. 10;

FIG. 16 represents an analysis performed with the device illustrated in FIG. 1 operating according to a sequence with six returns, for calibration samples comprising more or less salt;

FIG. 17 presents the areas of the peaks of FIG. 16, as a function of the NaCl concentration in the sample;

FIG. 18 represents the result of an analysis performed with the device illustrated in FIG. 1 operating according to a sequence with six returns, for previously purified cfDNA samples comprising more or less salt;

FIG. 19 represents changes in the area of the first peak and second peak of the curves in FIG. 18 as a function of the NaCl concentration, with the device illustrated in FIG. 1 operating according to a sequence with six returns;

FIG. 20 shows the result of the analysis of a plasma with a sequence comprising seven returns with change of buffer;

FIG. 21 shows the electrical current obtained during the step of concentration at pH 8 of the analysis illustrated in FIG. 20, with seven returns;

FIG. 22 shows the electrical current obtained during the last concentration step of the analysis illustrated in FIG. 20 with a change of buffer;

FIG. 23 shows the comparison of migrations of a calibration sample by itself or in the plasma, in a method with seven returns;

FIG. 24 shows the comparison of migrations of a calibration sample by itself or in the plasma, in a method with seven returns;

FIG. 25 represents an analysis of a circulating DNA sample directly from plasma with a fluorescence curve;

FIG. 26 represents a concentration profile according to size for the analysis illustrated in FIG. 25;

FIG. 27 represents an analysis of the same circulating DNA sample as that in FIGS. 25 and 26, after purification of the DNA from the plasma with a fluorescence curve;

FIG. 28 represents a concentration profile according to size for the analysis illustrated in FIG. 27;

FIG. 29 represents an analysis result with a concentration/separation method with no returns on large DNA fragments;

FIG. 30 represents an analysis result with a concentration/separation method with one return on large DNA fragments;

FIG. 31 represents a microfluidic device for utilising the method that is the subject of the invention; and

FIG. 32 illustrates, in the form of a logic diagram, steps in a particular embodiment of the method that is the subject of the invention.

### DESCRIPTION OF THE EMBODIMENTS

The present description is given in a non-limiting way, in which each characteristic of an embodiment can be combined with any other characteristic of any other embodiment in an advantageous way.

Note that the figures are not to scale.

In some embodiments, all or part of the means and variants of the devices disclosed in international application WO/2017/009566 are configured to utilise the method that is the subject of the invention. In other words, in these embodiments, the structural characteristics of the devices disclosed, for example with regard to the dimensions of the capillary equipped with a local restriction, can be transposed to the method that is the subject of the invention.

In some embodiments, the analysis buffer is a viscoelastic fluid. Preferably, such viscoelasticity is obtained by added a neutral polymer. More preferably, such a neutral polymer is used in the analysis buffer with a mass concentration between 0.1 and 10%. For example, such a neutral polymer is chosen from polyvinylpyrrolidone (PVP), poly (ethylene oxide) (PEO) and polyacrylamide (PAM) or mixtures thereof.

In some variants, the analysis buffer is not a viscoelastic fluid, and in particular contains no neutral polymer. Preferably, such variants are applied to samples comprising large nucleic acid fragments, for example having a size greater than or equal to 1000 bp.

In some embodiments, the capillary has a diameter, upstream from the local restriction, of at least 100 μm and preferably at least 300 μm.

In some embodiments, the diameter of the capillary upstream from the local restriction is between 300 μm and 600 μm.

In some embodiments, the local restriction has a cross-section of at least 2 μm, preferably at least 20 μm, and more preferably at least 40 μm. Preferably, such a cross-section is less than 100 μm.

In some embodiments, the device has a plurality of local restrictions. Preferably, such local restrictions have similar dimensions. For example, the device has:
a capillary having a diameter, upstream from the restrictions, between 0.5 and 5 mm; and
a plurality of local restrictions, such restrictions being similar to channels, with a diameter comprised between 2 and 50 μm.

In some embodiments, when the capillary and the restriction or restrictions have a circular geometry, the ratio of:
the diameter of the cross-section of the capillary upstream from the restriction, to
the diameter of the cross-section of the restriction, or the sum of the diameters of the cross-sections of the restrictions is between 5 and 20. In other words, the ratio of:
the area of the cross-section of the capillary upstream from the restrictions, to
the area of the cross-section of the restriction, or the sum of the areas of the cross-sections of the restrictions is between 25 and 400.

Thus, such ratios ensure not only an optimum retention of small-size nucleic acids during the concentration and desalting, and also ensures the concentration of larger nucleic acids, referred to as large-size, without obstruction.

In some embodiments, the capillary is a channel of a microfluidic chip equipped with a restriction. Examples of dimensions of channels and the restriction, applicable to these embodiments, are mentioned in the following scientific publications:
Teillet et al., “Characterization and minimization of band broadening in DNA electrohydrodynamic migration for enhanced size separation”, Soft Matter, 2020, 16, 5640-5649; and
Tijunelyte et al., “Hybridization-based DNA biosensing with a limit of detection of 4 fM in 30 s using an electrohydrodynamic concentration module fabricated by grayscale lithography”, Biomicrofluidics 16, 2022, 044111.

The microfluidic chip embodiments have a smaller geometry, in particular a shorter length, compared to other embodiments with no microfluidic chip mentioned in the present description.

In these embodiments, the microfluidic chip comprises the capillary referred to as the “wide capillary” or “wide channel”, a constriction, and then a capillary referred to as the “narrow capillary” or “narrow channel” that has a dimension smaller than the wide channel. In these embodiments, the assembly comprising the constriction and the narrow capillary forms the restriction. It is noted that, in general but non-restrictively, the channels of a microfluidic chip respectively have a rectangular cross-section.

In some embodiments, the wide channel has a width of at least 50 μm, and more preferably at least 400 μm.

In some embodiments, the narrow channel has a width of at least 2 μm, and more preferably at least 10 μm.

In some embodiments, the wide channel, the constriction and the narrow channel have a depth between 2 and 10 μm.

In some embodiments, the wide channel, the constriction and the narrow channel have a constant depth.

In some embodiments, the wide channel, the constriction and the narrow channel have variable, or referred to as “non-constant”, depths. Preferably, the depth of the wide channel upstream from the constriction is greater than the depth of the constriction and/or narrow channel. As a result, such a variation in depth, combined with the variation in the width of the channels and the constriction, ensures a larger increase in the ratio of the cross-section of the wide channel to the cross-section of the narrow channel, compared to a single variation in the width of the channels and the constriction with a constant depth.

In some embodiments, a method for forming the channels and constriction of a microfluidic chip comprises at least one step of etching a silica and/or glass wafer.

In some embodiments, the etching step comprises at least one step implementing a photolithography technique combined with a plasma etching technique, known in electronics for producing printed circuits. Preferably, such etching is carried out on a silica wafer. It is noted that an example of such an etching step is mentioned in the scientific publication by Teillet et al. cited previously in the present description.

For example, following such etching, the following capillaries are obtained:
a capillary corresponding to the wide channel, having a width equal to 800 μm; and
a capillary corresponding to the narrow channel downstream from the restriction, having a width equal to 15 μm; the capillaries and the constriction having a constant depth equal to 2 μm.

In some variants, the etching step comprises at least one step implementing a technique referred to as “grayscale” lithography combined with a plasma etching technique. It is noted that such an etching step makes it possible in particular to form the wide channel, constriction and narrow channel with variable depths. It is noted that an example of such an etching step is mentioned in the scientific publication by Tijunelyte et al. cited previously in the present description.

In some embodiments, during the implementation of the method that is the subject of the invention, nucleic acids are detected, for example, by using fluorescence detectors or UV adsorption detectors known to the person skilled in the art. When a fluorescence detector is used, the nucleic acids are, for example, chemically tagged with a fluorescent probe or tagged using a DNA intercalant or groove binder. It is noted that these different taggings are known to the person skilled in the art.

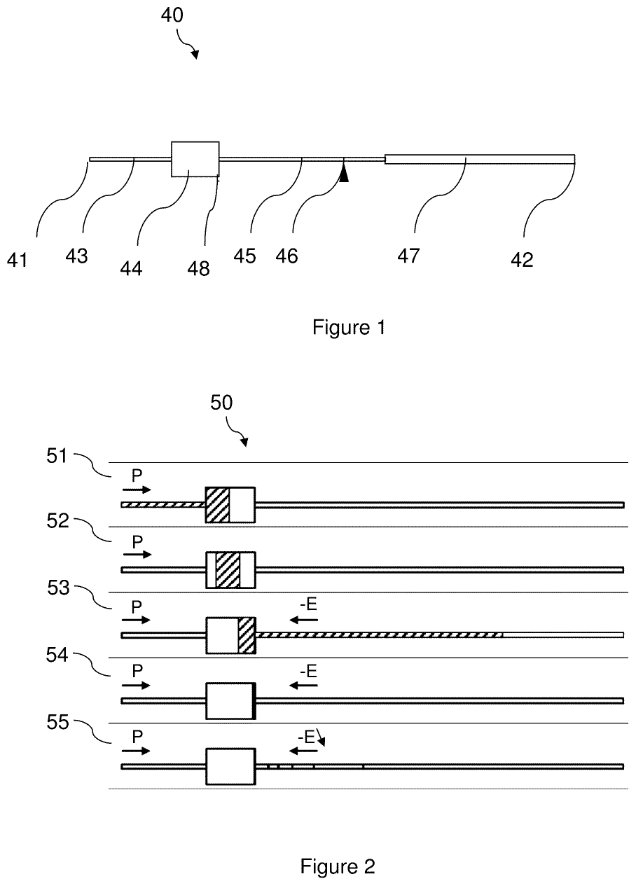

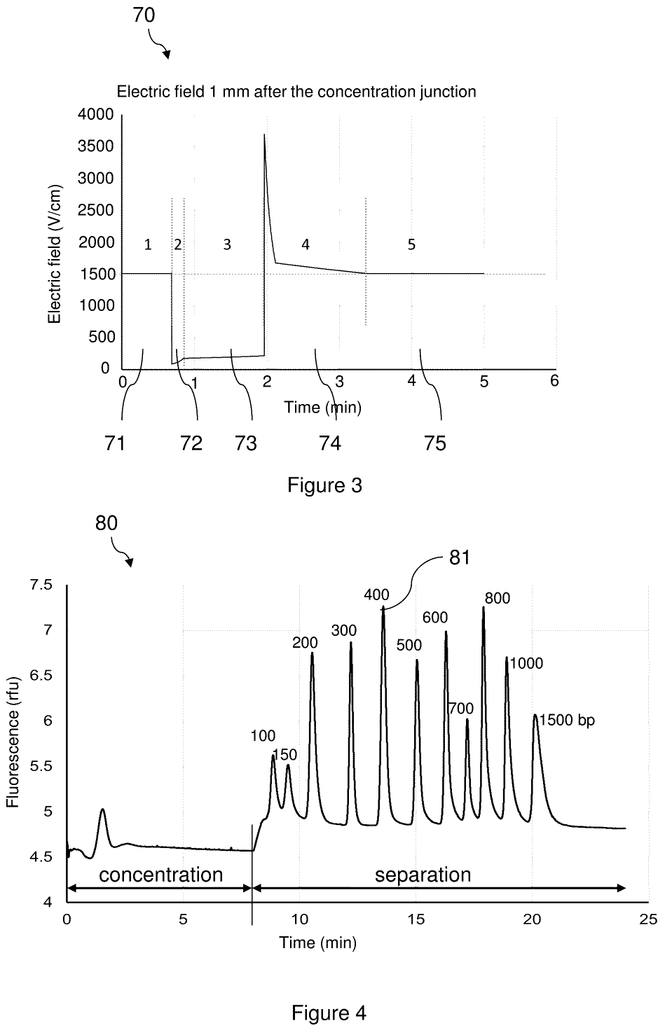

Examples 1 and 2, described with reference to FIGS. 1 to 9, describe the experimental results of a device 40 of the prior art operating according to the sequence illustrated in FIG. 2, a sequence also called “O returns”.

### Example 1: “0 Returns” Method on Device 40, Sample without Salts

Equipment used:
Agilent CE G1600 (registered trademark);
Picometrics detector (registered trademark), Zetalif LED480 (registered trademark) (PMT set to 630V, RT set to 0.5s);
Chemical products:

TABLE 1

| D [symbol] signation | R [symbol] f [symbol] rence | Fournisseur |
|---|---|---|
| Eau | BP2819-1 | FISHER BioReagents |
| | | (marque d [symbol] pos [symbol] e) |
| NaOH | 28244295 | ProLabo (marque d [symbol] pos [symbol] e) |
| HCl | 317.1000 | MERCK (marque d [symbol] pos [symbol] e) |
| Tampon d'analyse | 16-BB-DNA1K/10 | Adelis Technologies |
| DNA1K | | (marque d [symbol] pos [symbol] e) |
| Solution de | 16-BB-DNA/09 | Adelis Technologies |
| conditionnement | | |
| Echantillon [symbol] talon | 16-BB-DNA1K/08 | Adelis Technologies |
| 1K | | |

[symbol]

indicates data missing or illegible when filed

A “DNA 1K analysis” is a profiling of DNA sizes in a range 100 bp to 1500 bp (therefore, of the order 1 kb, hence the designation 1K). It is essentially defined by the analysis buffer and the analysis method. The analysis method is essentially defined by the pressure/voltage timeline to carry out the concentration and then the separation, with cleaning/conditioning methods described in the examples below. Similarly, a “DNA 10K analysis” is the profiling of DNA sizes in the range 1 kbp to 10 kbp.

The term “DNA 1K buffer” refers to the analysis buffer for carrying out this “DNA 1K” analysis. The term “DNA 1K calibration sample” refers to the calibration sample that serves as a calibration for DNA 1K analyses. It is also called the “DNA 1K ladder”, or the “calibration 1K sample”.

As described below, preferably, the number of returns depends on the salinity of the samples. For example, two types of analysis are defined, one for the samples with no or low salt content, the other for samples with salts. Therefore:
the “LoSalt” DNA 1K analysis uses the method with two returns as shown in example 3; it is recommended for samples between 0 and 15 mM NaCl; and
the “HiSalt” DNA 1K analysis uses the method with six returns as shown in example 4; it is recommended for samples up to 130 mM NaCl.

These two analyses use the same DNA 1K analysis buffer, and the same calibration 1K sample. They differ solely in the number and timeframe of the returns present during the concentration phase.

The calibration 1K sample contains DNA fragments with the following sizes: 100 bp, 150 bp, 200 bp, 300 bp, 400 bp, 500 bp, 600 bp, 700 bp, 800 bp, 1000 bp, and 1500 bp.

The 0 returns method for the device 40 is described in table 2 below.

TABLE 2
0 returns method for the device 40

| | | Pression | Voltage | Dur [symbol] e |
|---|---|---|---|---|
| Intet | Outlet | (Bar) | (kV) | (min) |
| A/Pr [symbol] - | | | | |
| conditionnement | | | | |
| HCl 0.1M | Poubelle | 10 | 0 | 2 |
| Conditionnement | Poubelle | 10 | 0 | 4 |
| Tampon | Poubelle | 10 | 0 | 6 |
| d'analyse | | | | |
| B/Injections | | | | |
| (op [symbol] ration 51) | | | | |
| Echantillon | Poubelle | 5 | 0 | 1.33 (80s) |
| C/Transfert | | | | |
| (op [symbol] ration 52) | | | | |
| Tampon | Tampon | 5 | 0 | 0.17 (10s) |
| d'analyse | d'analyse | | | |
| D/Concentration | | | | |
| (op [symbol] rations | | | | |
| 53 et 54) | | | | |
| Tampon | Tampon | 10 | 25 | 8 |
| d'analyse | d'analyse | | | |
| E/Separation | | | | |
| (op [symbol] ration 55) | | | | |
| Tampon | Tampon | 10 | de 25 [symbol] 10 | 0.4 |
| d'analyse | d'analyse | | | |
| | | 10 | de 10 [symbol] 5 | 0.4 |
| | | 10 | de 5 [symbol] 0.6 | 3.2 |
| | | 10 | de 0.6 [symbol] 0.1 | 11 |
| | | 10 | de 0.1 [symbol] 0 | 0.1 |
| | | 10 | 0 | 1.9 |

[symbol]

indicates data missing or illegible when filed

FIG. 4 shows an analysis result 80 for a calibration DNA sample without salts: fluorescence curve for a calibration DNA sample without salts according to a DNA 1K analysis, “0 returns” method. Each fluorescence peak 81 corresponds to the DNA size indicated on the graph in base pairs (bp).

### Example 2: “0 Returns” Method on Device 40, Sample with Salts

The equipment and reagents are identical to those described for example 1. The range of salts in the calibration 1K sample were produced as follows:

TABLE 3

| | | | | Volume |
|---|---|---|---|---|
| | | Volume NaCl | Volume TE | Echantillon |
| Concentration sels | | 1M (μl) | 1X (μl) | (μl) |
| 0 | mM | 0 | 13 | 87 |
| 10 | mM | 1 | 12 | 87 |
| 15 | mM | 1.5 | 11.5 | 87 |
| 20 | mM | 2 | 11 | 87 |
| 50 | mM | 5 | 8 | 87 |
| 100 | mM | 10 | 3 | 87 |
| 130 | mM | 13 | 0 | 87 |

For preparing the range of salts with a circulating DNA sample, the sample volume available being only 140 μL, the range was produced as follows:

TABLE 4

| | | | | Volume |
|---|---|---|---|---|
| | | Volume NaCl | Volume TE | Echantillon |
| Concentration sels | | 1M (μl) | 1X (μl) | (μl) |
| 0 | mM | 0 | 2.6 | 17.4 |
| 10 | mM | 0.2 | 2.4 | 17.4 |
| 15 | mM | 0.3 | 2.3 | 17.4 |
| 20 | mM | 0.4 | 2.2 | 17.4 |
| 50 | mM | 1 | 1.6 | 17.4 |
| 100 | mM | 2 | 0.6 | 17.4 |
| 130 | mM | 2.6 | 0 | 17.4 |

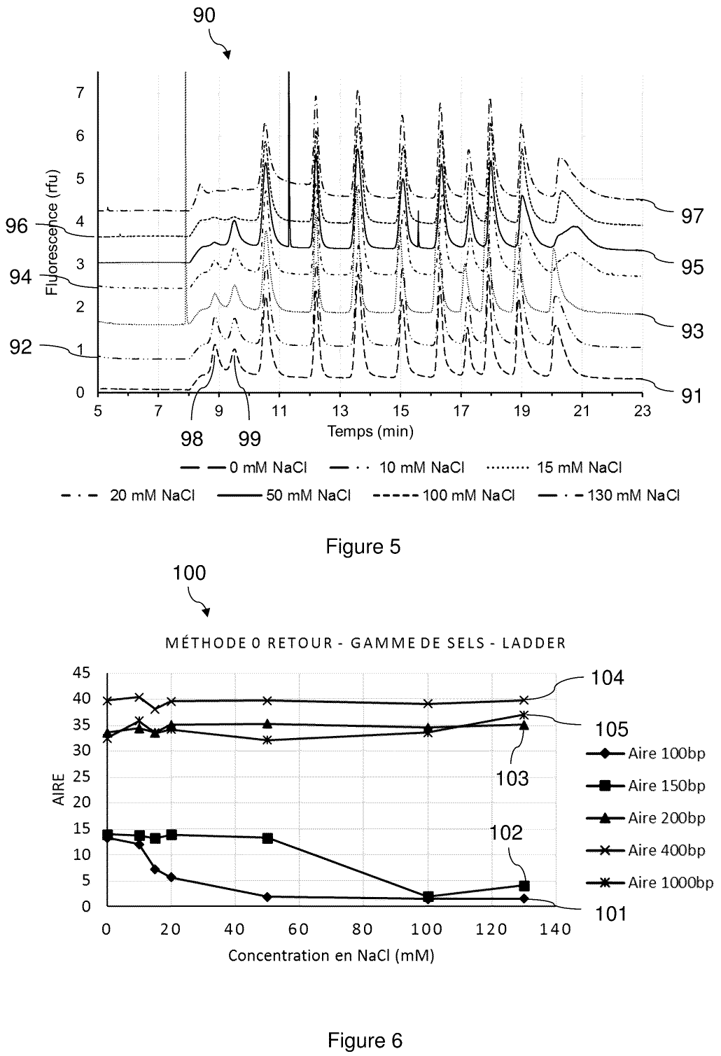

The “O returns” method for the device 40 is the same as in example 1. FIG. 5 shows the result 90 for calibration DNA samples containing more or less salt. This analysis with the “0 returns” method for samples containing 0 mM NaCl is shown by curve 91, 10 mM NaCl by curve 92, 15 mM NaCl by curve 93, 20 mM NaCl by curve 94, 50 mM NaCl by curve 95, 100 mM NaCl by curve 96, and 130 mM NaCl by curve 97.

The fluorescence values have been separated on the vertical axis of fluorescence by adding a constant to facilitate the display of the curves.

FIG. 5 shows that the 100 bp peak 98, corresponding to a little less than nine minutes on the time axis, is present in the samples containing 0 and 10 mM NaCl, is lower in the samples containing 15 or 20 mM NaCl, and is almost absent for the higher salt concentrations. Similarly, the 150 bp fragment 99 is present up to a concentration of approximately 50 mM, and then disappears. FIG. 6 presents the areas 100 of the peaks as a function of the NaCl concentration in the sample, for the peaks 100 bp by curve 101, 150 bp by curve 102, 200 bp by curve 103, 400 bp by curve 104, and 1000 bp by curve 105, and as a function of the concentration of salts in the DNA 1K buffer with the “0 returns” method.

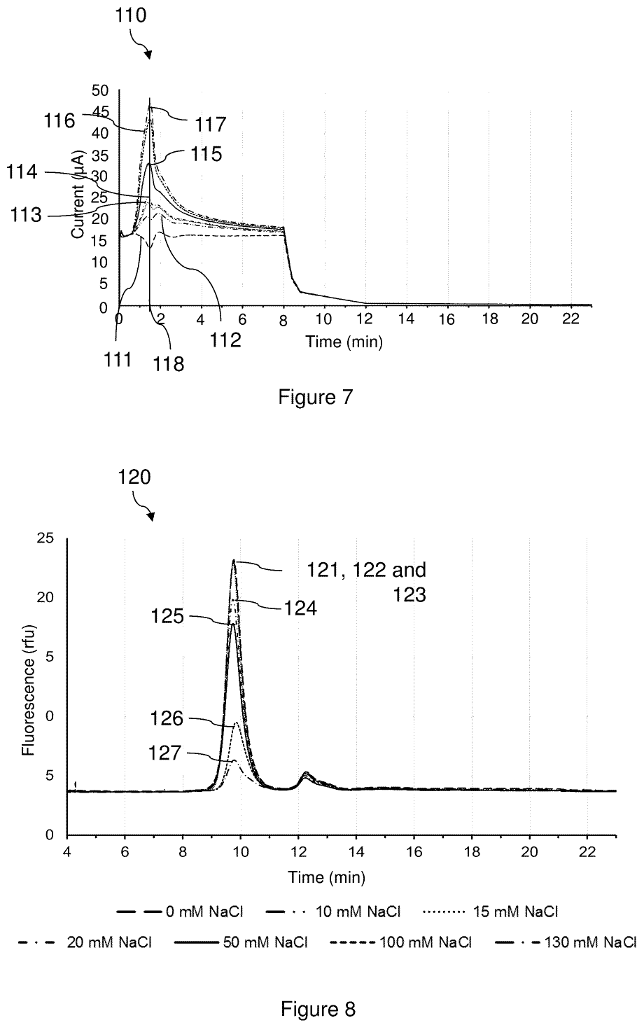

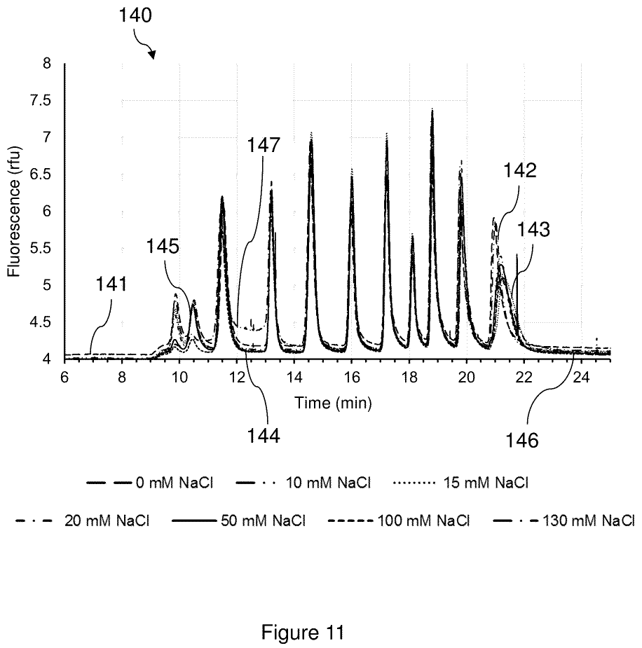

Another lesson drawn from these experiments come from the intensity 110 of the electrical current observed during the concentration, and presented in FIG. 7. We observed that the intensity of the peak 118 of current during the concentration phase, at about 1.5 minutes, provides information on the concentration of salts for the sample, the curves 111 to 117 corresponding, respectively, to the concentrations of curves 91 to 97 in FIG. 5. This measurement can be used as a quality control for the analysis, to notify the user when the conductivity of the sample is too high for a reliable analysis of his sample by the 0 returns method. This measurement can also be used to adjust the number and timeframe of the returns in the method that is the subject of the invention, described with reference to FIGS. 11 and following, as a function of the salinity of the sample. The measurement of the current, or the measurement of the derivative of the current, can therefore be used to cause an earlier return, when the salinity is high.

For a circulating DNA sample containing more or less salt, a range of salts (from 0 mM to 130 mM) was produced on a circulating DNA sample with the “0 returns” method, as shown in FIG. 8. FIG. 8 shows the analysis 120 with the “O returns” method for a purified circulating DNA sample containing 0 mM NaCl, curve 121; 10 mM NaCl, curve 122; 15 mM NaCl, curve 123; 20 mM NaCl, curve 124; 50 mM NaCl, curve 125; 100 mM NaCl, curve 126; and 130 mM NaCl, curve 127.

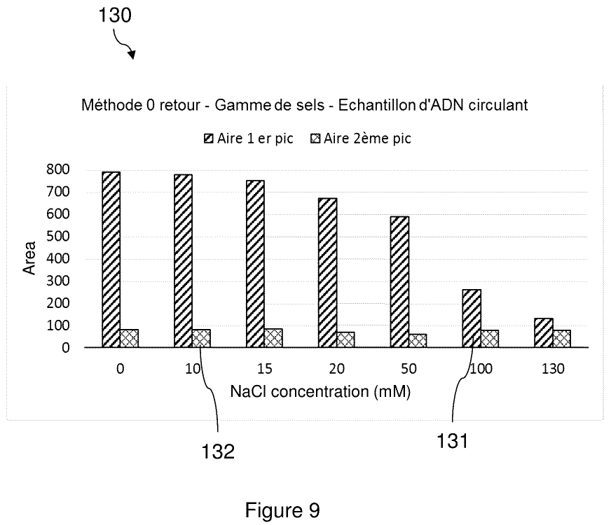

FIG. 9 shows the changes 130 in the area 131 of the first peak, on the left, and the area 132 of the second peak, on the right, as a function of the NaCl concentration with the “0 returns” method. The values come from FIG. 8. It can be seen that, with the “O returns” method, the area of the second peak is stable regardless of the concentration of salts in the sample. In contrast, the area of the first peak is stable up to 10-15 mM, and then decreases significantly. With 130 mM of salts in the sample, only 16% of the first peak 131 is retained during the step of concentration.

Examples 3 to 9 show results obtained by the utilisation of the present invention. Throughout the description, it is experiments carried out with samples containing DNA that are described. However, the present invention is not limited to this type of nucleic acid but, on the contrary, extends to other nucleic acids, for example single-stranded RNA or DNA.

In example 3, shown with reference to FIGS. 11 to 15, by inserting two returns during the concentration step, it becomes possible to analyse small-size DNA samples containing up to 15 mM of salts, i.e. a content 50% higher than what is possible with the 0 returns method. Above 15 mM of salts, the small fragments are no longer retained correctly. FIG. 13 displays the time positioning of two return periods, 161 and 162, in the timeline of the method. During these returns, because no electric voltage is applied, the electrical current is zero.

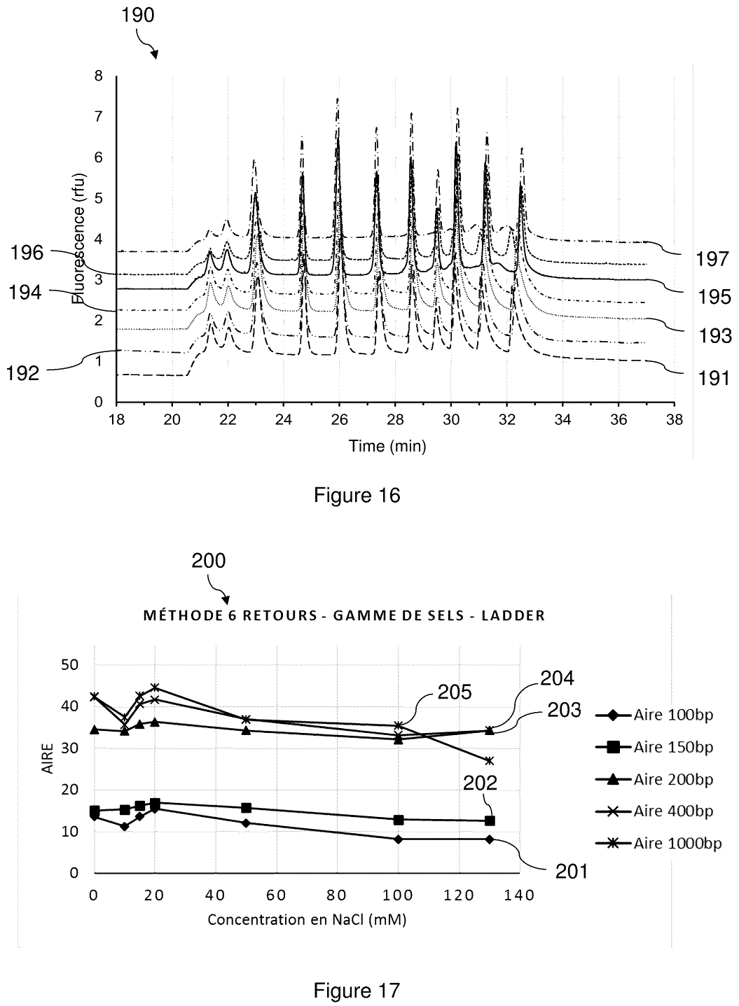

In example 4, shown with reference to FIGS. 16 and 17, the introduction of six returns during the concentration phase enables the full retention of all the fragments of 100 bp to 1500 bp up to 130 mM NaCl, nevertheless with a slight drop in intensity for the fragments of 100 and 150 bp beyond 100 mM NaCl. In the same way, with these six returns, the main peak of circulating DNA is retained up to an NaCl concentration of 130 mM, with a reduction in intensity of only approximately 15%.

During the analysis of the results of an unknown DNA sample, the fluorescence profile is converted into a concentration profile thanks to the fluorescence profile of the calibration sample, for which the concentration is known for each DNA fragment length. The slight drop in intensity for the smallest DNA fragments is therefore compensated for by the calibration based on the profile of the calibration sample.

In a human biological sample, there is not only a salinity equivalent to approximately 130 mM NaCl, but also, in most cases, proteins. Some of these proteins are positively charged. Therefore, they attach themselves to the walls of the capillary and also to the DNA, which are negatively charged. This results in DNA sticking to the walls, which adhesion is detrimental to the analysis.

To avoid this phenomenon, it is preferable to carry out the concentration at high pH, so that the overwhelming majority of the proteins are negatively charged, and the DNA sticking to the walls via the proteins disappears. Unfortunately, at a high pH, the quality of the DNA separation is not as good as with a neutral or slightly acidic pH, no doubt because an electro-osmosis phenomenon that is too intense. That is why, in some embodiments, a method with returns and change of buffer is utilised to analyse the circulating DNA directly in the plasma. First of all a concentration at high pH with returns takes place, which allows the salts and protein residues to be removed from the sample. Then, after a last return, a new concentration phase takes place, this time at a neutral or slightly acidic pH, more favourable for separation. This concentration phase allows the buffer of the sample to be changed. In a third step the separation, identical to the separation in the other methods described in examples 1 and 2, takes place.

This method with return(s) and change of buffer is described in example 5. It makes it possible to obtain a profile of circulating DNA directly from the plasma, without prior purification of the DNA. The first part of the concentration, at high pH, takes place at 11 bars. The second part of the concentration, and the separation, take place at a slightly acidic pH and 7 bars to improve the quality of the DNA separation.

This method makes it possible to reduce, even eradicate, the possible effect of the matrix of the biological sample on the DNA separation profile. Example 6 therefore shows how a calibration DNA migrates in the same way in a plasma and in a buffer without salts.

In example 7, we confirm the reliability of the method that is the subject of the invention by comparing size profiles of circulating DNA, obtained either by the method of example 3 (two returns) after purification of the DNA, or by the method of example 5 directly on plasma (method with seven returns).

In example 8, we show that the return principle also works on DNA with larger sizes, between 1 and 10 kb. It is clear that this return principle will also work on even larger DNA, for example up to 200 kb.

All the above examples correspond to experiments carried out with the device 40 developed by Adelis (registered trademark), which uses a capillary device (FIG. 1) installed in an Agilent capillary electrophoresis instrument.

The technology also works in a microfluidic chip format, and the present invention can equally be utilised to analyse DNA samples with salts, or to analyse DNA directly in biological fluids, using microfluidic chips. Example 9 describes such a microfluidic chip.

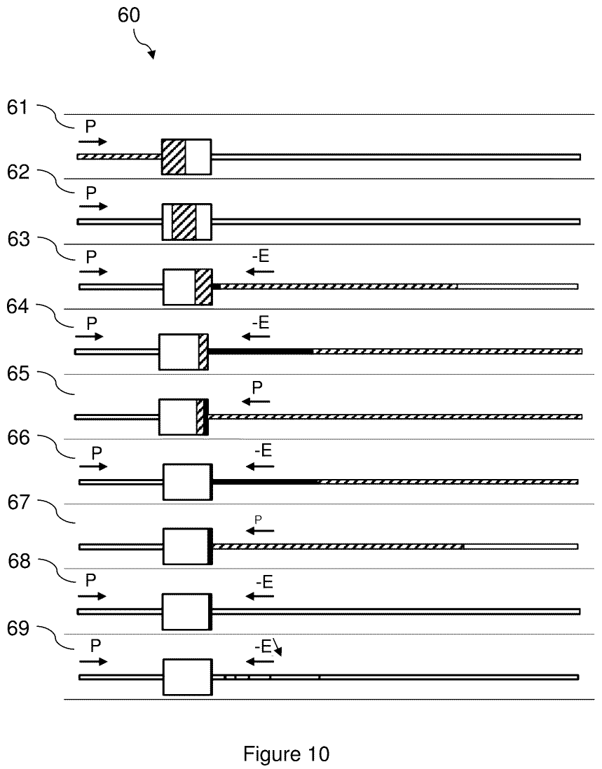

FIG. 10 shows a typical sequence of operation of a DNA analysis with the method that is the subject of the invention implemented with two returns and the device 40. The distal capillary 47 is not shown in this figure. FIG. 10 shows the concentrated DNA in black, and the salts in cross-hatching.

During an operation 61, the sample is injected into the device, by applying pressure for a given duration.

During an operation 62, the injected sample is pushed by pressure to the centre of the injection chamber 44.

During operations 63 and 64, the DNA is concentrated at the concentration junction 48 of the injection chamber 44, by applying a first pressure causing a flow in a first direction of flow (from left to right in FIG. 10) and a first difference in electric potential whose action on the nucleic acid molecules is opposite to the first direction of flow and causes the retention or deceleration of the nucleic acid molecules in the capillary. The separation capillary 45 fills with the sample solution, except for the largest DNA fragments, which remain retained at the concentration junction 48.

The migration speed of the smallest DNA fragments is slower than the average speed of the flow. In contrast, the ions forming the salts are too small for the development of a transverse force pushing them towards the wall, and they advance at the average speed of the flow, greater or less than their speed of electrophoresis depending on their positive or negative charge.

During an operation 65, a flow of the sample, in a second direction of flow opposite to the first direction of flow, is caused by applying a second pressure in the opposite direction to the pressure exerted on the sample solution. A portion of the sample solution that had entered the separation capillary 45 flows back into the injection chamber 44, carrying DNA fragments which therefore return into the injection chamber 44. In the description, the operation 65 is called a “return”.

Therefore, the concentration operation is stopped before the smallest fragments arrive at the end of the separation capillary, and a second pressure is applied in the opposite direction to the first pressure to take the volume from the separation capillary 45 back into the injection chamber 44.

During an operation 66, the DNA is again concentrated at the concentration junction 48 of the injection chamber 44, by applying the first pressure causing a flow in the first direction of flow and the first difference in electric potential whose action on the nucleic acid molecules is opposite to the first direction of flow and causes the retention of nucleic acid molecules in the capillary. The separation capillary 45 fills with the sample solution, except for the largest DNA fragments, which remain retained at the concentration junction 48. Thus a new concentration step is carried out.

During a second return operation 67, a flow of the sample, in the second direction of flow, is caused by applying a second pressure in the opposite direction to the pressure exerted on the sample solution. A portion of the sample solution that had entered the separation capillary 45 flows back into the injection chamber 44, carrying DNA fragments which therefore return into the injection chamber 44.

During an operation 68, the DNA is again concentrated at the concentration junction 48 of the injection chamber 44, by applying the first pressure causing a flow in the first direction of flow and the first difference in electric potential whose action on the nucleic acid molecules is opposite to the first direction of flow and causes the retention of nucleic acid molecules in the capillary. The separation capillary 45 fills with the sample solution.

Therefore, this alternation of return and concentration phases is reiterated. The salts are therefore evacuated progressively from the device, while the DNA molecules are mainly retained. The small DNA fragments can therefore be analysed correctly, which was impossible with the devices and methods of the prior art.

During an operation 69, the DNA is separated according to size by a progressive decrease in the electric field applied between the inlet 41 and the outlet 42, the pressure generally being maintained constant.

Typically, during the concentration steps 63, 64, 66 and 68, the following physical values are applied to retain DNA fragments as small as 100 bp:
voltage: 25,000 V
pressure: 10 bars
the solution's viscosity and resistivity in the capillary are respectively 40 mPa·s and 12.4 Ω.m at 25° C.

In the separation capillary 45, the following are obtained:
an electric field 100 to 1500 V/cm, depending on the resistivity of the sample; and
an average fluid speed of 9 mm/s.

Preferably, during the steps 65 and 67 of the laminar flow of the sample in a second direction of flow, the sample is subjected to a second difference in electric potential whose action on the nucleic acid molecules is opposite to the first direction of flow. The second difference in potential can therefore be identical to the first difference in potential. The molecules still close to the walls of the separation capillary therefore return more rapidly into the injection chamber than during a return by pressure alone, as described above.

### Example 3: Two Returns Method, for Calibration DNA Samples Containing More or Less Salt

A method implementing two returns in a device 40 is described below.

The equipment and methods are identical to those of examples 1 and 2.

The method is numerically described in the following table.

TABLE 5
2 returns method for the device 40

| | | Pression | Voltage | Dur [symbol] e |
|---|---|---|---|---|
| Inlet | Outlet | (Bar) | (kV) | (min) |
| A/Pr [symbol] - | | | | |
| conditionnement | | | | |
| HCl 0.1M | Poubelle | 10 | 0 | 2 |
| Conditionnement | Poubelle | 10 | 0 | 4 |
| Tampon | Poubelle | 10 | 0 | 6 |
| d'analyse | | | | |
| B/Injection | | | | |
| (op [symbol] ration 61) | | | | |
| Echantillon | Poubelle | 5 | 0 | 1.33 (80s) |
| C/Transfert | | | | |
| (op [symbol] ration 62) | | | | |
| Tampon | Tampon | | 0 | 0.17 (10s) |
| d'analyse | d'analyse | | | |
| D/Concentration | | | | |
| et Retours | | | | |
| (op [symbol] rations | | | | |
| 63 [symbol] 68) | | | | |
| Tampon | Tampon | 10 | 25 | 2 |
| d'analyse | d'analyse | | | |
| | | −10 | 0 | 0. [symbol] |
| | | 10 | 25 | 2 |
| | | −10 | 0 | 0. [symbol] |
| | | 10 | 25 | 4 |

| E/Separation | | Pression | Voltage | Dur [symbol] e |
|---|---|---|---|---|
| (operation 69) | Outlet | (Bar) | (kV) | (min) |
| Tampon | Tampon | 10 | de 25 [symbol] 10 | 0.4 |
| d'analyse | d'analyse | | | |
| | | 10 | de 10 [symbol] 5 | 0.4 |
| | | 10 | de 5 [symbol] 0.6 | 3.2 |
| | | 10 | de 0.8 [symbol] 0.1 | 11 |
| | | 10 | de 0.1 [symbol] 0 | 0.1 |
| | | 10 | 0 | 1.9 |

[symbol]

indicates data missing or illegible when filed

The two passages at a negative pressure of −10 bars correspond to the two returns introduced during the concentration phase, operations 65 and 67 in FIG. 10, in accordance with the present invention.

A range of salts (from 0 mM to 130 mM) were produced with a calibration sample and analysed with the two returns method.

FIG. 11 shows the analysis result 140 with the two returns method for a calibration sample containing 0 mM NaCl in long regular dashed lines, curve 141; containing 10 mM NaCl in discontinuous lines alternating a dash and two dots, curve 142; containing 15 mM NaCl in discontinuous lines consisting of regularly spaced dots, curve 143; containing 20 mM NaCl in discontinuous lines alternating a short line and a dot, curve 144; containing 50 mM NaCl in a solid line, curve 145; containing 100 mM NaCl in short regular dashed lines, curve 146; and containing 130 mM NaCl in discontinuous lines alternating a long line and a dot, curve 147.

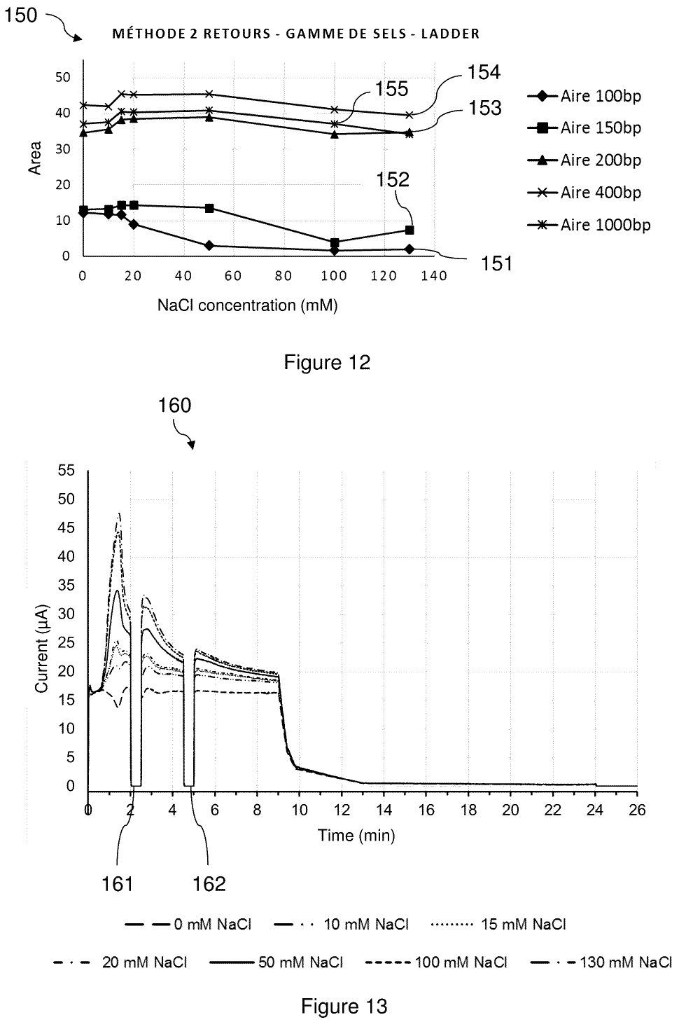

FIG. 12 presents the areas 150 of the peaks as a function of the NaCl concentration in the calibration sample for the peaks 100 bp by curve 151, 150 bp by curve 152, 200 bp by curve 153, 400 bp by curve 154, and 1000 bp by curve 155. The areas of the peaks 200 bp, 400 bp and 1000 bp remain stable regardless of the concentration of salts in the sample. The 100 bp area remains stable up to 15 mM NaCl, and then decreases significantly. The 150 bp area decreases beyond 50 mM.

Switching to a method with two returns therefore makes it possible to analyse DNA samples containing up to 15 mM of salts in the sample.

The profiles 160 of the current during the analyses are given in FIG. 13. The passages at zero current 161 at minute 2 and 162 at minute 4.5 are the two returns that were introduced during the concentration phase, in accordance with the invention.

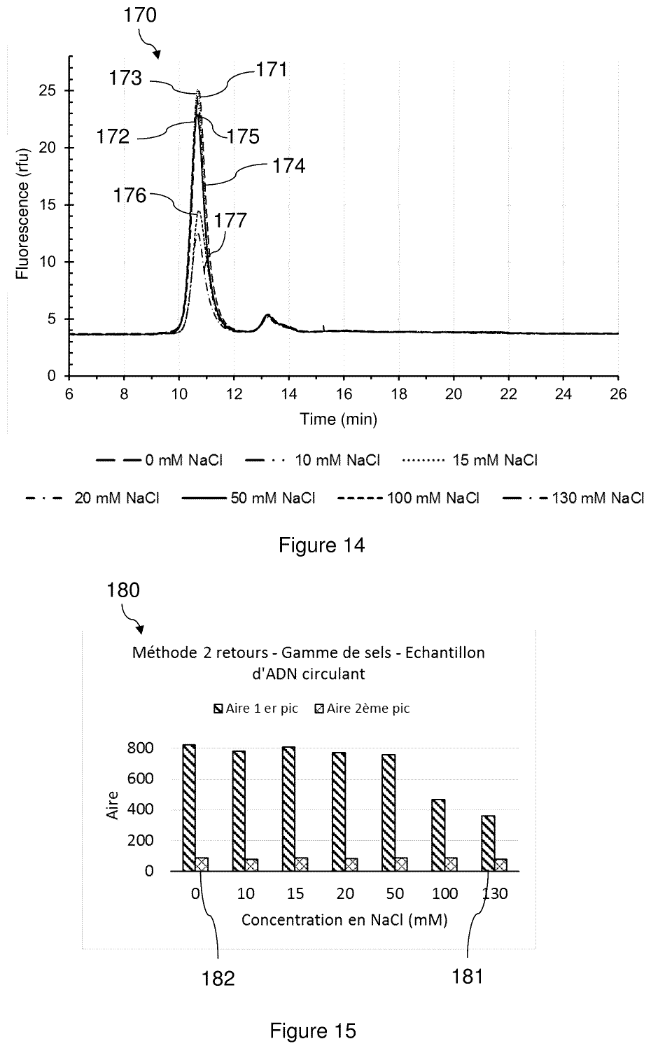

We obtain equivalent results with a sample of circulating DNA. FIG. 14 shows the analysis result 170 with the two returns method for a purified circulating DNA sample containing 0 mM NaCl in long regular dashed lines, curve 171; containing 10 mM NaCl in discontinuous lines alternating a dash and two dots, curve 172; containing 15 mM NaCl in discontinuous lines consisting of regularly spaced dots, curve 173; containing 20 mM NaCl in discontinuous lines alternating a short line and a dot, curve 174; containing 50 mM NaCl in a solid line, curve 175; containing 100 mM NaCl in short regular dashed lines, curve 176; and containing 130 mM NaCl in discontinuous lines alternating a long line and a dot, curve 177.

FIG. 15 shows the changes 180 in the area 181 of the first peak, on the left in each pair of areas, and the area 182 of the second peak, on the right, as a function of the NaCl concentration with the two returns method. The values come from FIG. 14. With the two returns method, the area 182 of the second peak is stable regardless of the concentration of salts in the sample. The area 181 of the first peak is stable up to 15 mM, and then decreases slightly to 50 mM. With 130 mM of salts in the sample, 43% of the first peak is retained during the step of concentration.

### Example 4: Method with Six Returns

The equipment and methods are identical to those described in the above examples.

The six returns method is numerically described in the following table, it being noted that the steps of pre-conditioning A, injecting the sample B, and then transferring the sample C into the injection chamber, are similar to the 0 returns method.

TABLE 6
Six returns method for a device 40

| | | Pression | Voltage | Dur [symbol] e |
|---|---|---|---|---|
| | Outlet | (Bar) | (kV) | (min) |
| D/Concentration | | | | |
| et Retours | | | | |
| Tampon | Tampon | 10 | 25 | 1 |
| d'analyse | d'analyse | | | |
| | | −10 | 0 | 0.75 |
| | | 10 | 25 | 1 |
| | | −10 | 0 | 0.75 |
| | | 10 | 25 | 1 |
| | | −10 | 0 | 0.75 |
| | | 10 | 25 | 2 |
| | | −10 | 0 | 0.75 |
| | | 10 | 25 | 3 |
| | | −10 | 0 | 0.5 |
| | | 10 | 25 | 4.5 |
| | | −10 | 0 | 0.5 |
| | | 10 | 25 | 4 |
| E/S [symbol] peration | | | | |
| Tampon | Tampon | 10 | de 25 [symbol] 10 | 0.4 |
| d'analyse | d'analyse | | | |
| | | 10 | de 10 [symbol] 5 | 0.4 |
| | | 10 | de 5 [symbol] 0.6 | 3.2 |
| | | 10 | de 0.6 [symbol] 0.1 | 11 |
| | | 10 | de 0.1 [symbol] 0 | 0.1 |
| | | 10 | 0 | 1. [symbol] |

[symbol]

indicates data missing or illegible when filed

To obtain a result for calibration DNA samples containing more or less salt, a range of salts (from 0 mM to 130 mM) were produced with the calibration 1K sample, using the same method as in example 2. FIG. 16 shows the analysis result 190 with the six returns method for a calibration 1K sample containing 0 mM NaCl in long regular dashed lines, curve 191; 10 mM NaCl in discontinuous lines alternating a dash and two dots, curve 192; 15 mM NaCl in discontinuous lines consisting of regularly spaced dots, curve 193; 20 mM NaCl in discontinuous lines alternating a short line and a dot, curve 194; 50 mM NaCl in a solid line, curve 195; 100 mM NaCl in short regular dashed lines, curve 196; and 130 mM NaCl in discontinuous lines alternating a long line and a dot, curve 197.

FIG. 17 shows changes in the area for the peaks 100 bp by curve 201, 150 bp by curve 202, 200 bp by curve 203, 400 bp by curve 204, and 1000 bp by curve 205, as a function of the NaCl concentration in the sample with the six returns method. The area of the peaks is stable for all the peaks, up to 130 mM NaCl. However, it seems that the 100 bp peak drops slightly in intensity from 100 mM NaCl.

Switching to a method with six returns therefore makes it possible to analyse DNA samples containing up to 130 mM of salts in the sample.

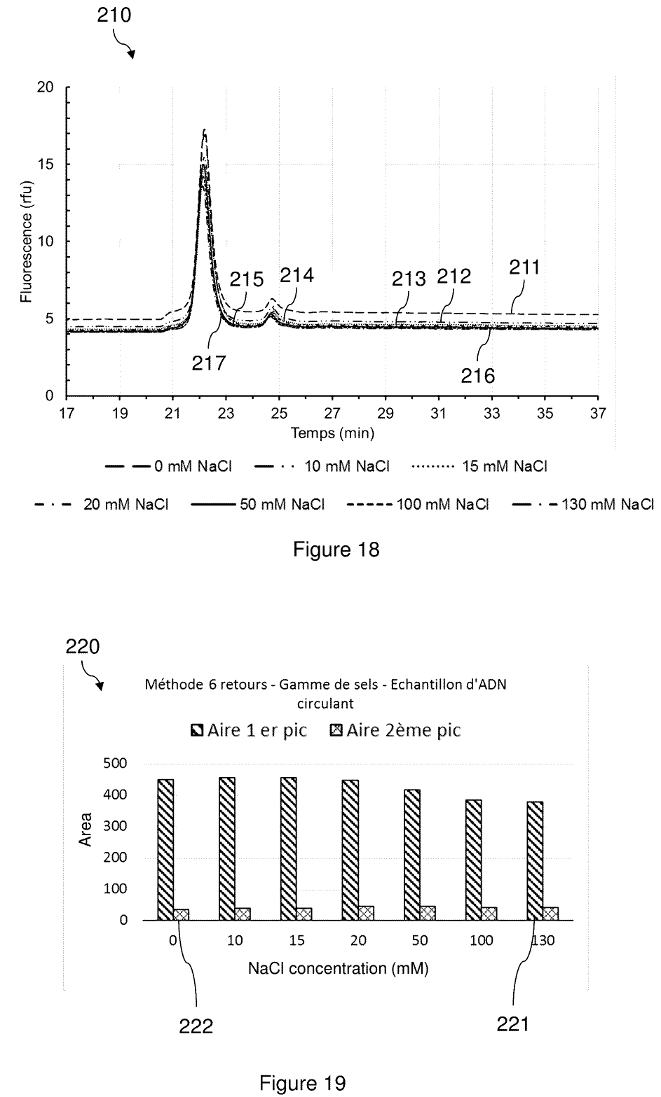

FIG. 18 shows the analysis results 210 for a purified circulating DNA sample containing more or less salt, with the six returns method for a sample containing 0 mM NaCl in long regular dashed lines, curve 211; mM NaCl in discontinuous lines alternating a dash and two dots, curve 212; 15 mM NaCl in discontinuous lines consisting of regularly spaced dots, curve 213; 20 mM NaCl in discontinuous lines alternating a short line and a dot, curve 214; 50 mM NaCl in a solid line, curve 215; 100 mM NaCl in short regular dashed lines, curve 216; and 130 mM NaCl in discontinuous lines alternating a long line and a dot, curve 217.

The graph 220 in FIG. 19 shows changes in the area 221 of the first peak, and in the area 222 of the second peak as a function of the NaCl concentration with the six returns method. The values come from FIG. 18.

It can be seen that the area 222 of the second peak is stable up to 130 m NaCl.

The area 221 of the first peak is stable up to 20-50 mM NaCl. At 100 and 130 mM, it shows a slight drop in intensity. This drop in intensity does not interfere with the analysis, since it is corrected when the fluorescence results are converted into concentration results thanks to the calibration based on the calibration sample.

### Example 5: Seven Returns Method for Direct Analysis of Plasma

The equipment and methods are identical to those of the above examples.

However, a first analysis buffer, with a basic pH, is added. In this example, a Tris-acetate mixture at pH 8 was used: Tris 20 mM, acetic acid 10 mM, EDTA (ethylenediamine tetra-acetic acid, a diamino tetracarboxylic acid) 0.5 mM, PVP (Polyvinylpyrrolidone) 5%, BSA (acronym for “bovine serum albumin”) 0.5 mg/mL.

Also, in this example the cleaning product RNAse Away (registered trademark) from ThermoFisher Scientific (registered trademark) was added to the conditioning step.

The plasma samples are pre-treated by proteinase K digestion in the presence of detergent, an operation intended to release the nucleic acids from the vesicles and nucleoprotein complexes in which they are usually trapped. This step of proteinase K digestion is currently performed as the first step in the methods for purifying the DNA of a biological sample. This involves simply checking that a sufficient level of proteinase K is used (the activity of the enzyme depends on the supplier and on the reference chosen), and choosing a non-ionic detergent (an ionic detergent results in an electro-osmosis and electrical current that are too high for the method).

In this example, 100 μL of plasma was pre-treated by proteinase K and only 15 μL of pre-treated plasma was placed in the injection vial of the Agilent CE. And approximately 1 μL of pre-treated plasma was injected by the Agilent CE into the device 40 and analysed by the 7 returns method.

### Description of the Seven Returns Method:

TABLE 7
Seven returns method and change of buffer for the device 40

| | | Pression | Voltage | Dur [symbol] e |
|---|---|---|---|---|
| Inlet | Outlet | (Bar) | (kV) | (min) |
| A/Pr [symbol] - | | | | |
| conditionnement | | | | |
| Poubelle | RRASE Away | 10 | 0 | 4 |
| RNASE Away | Poubelle | −10 | 0 | 4 |
| Water | Poubelle | 10 | 0 | 4 |
| HCl 0.1M | Poubelle | 10 | 0 | 2 |
| Conditionnement | Poubelle | 10 | 0 | [symbol] |
| Tampon pH 8 | Poubelle | 10 | 0 | 6 |
| B/Injection | | | | |
| Echantillon | Poubelle | 5 | 0 | 1.33 (80s) |
| C/Transfert | | | | |
| Tampon pH 8 | Tampon pH 8 | 5 | 0 | 0.17 (10s) |
| D/Concentration | | | | |
| et Retours | | | | |
| Tampon pH 8 | Tampon pH 8 | 11 | 25 | 1 |
| | | −11 | 0 | 0.75 |
| | | 11 | 26 | 1 |
| | | −11 | 0 | 0.75 |
| | | 11 | 25 | 1 |
| | | −11 | 0 | 0.75 |
| | | 11 | 25 | 2 |
| | | −11 | 0 | 0.75 |
| | | 11 | 25 | 3 |
| | | −11 | 0 | 0.5 |
| | | 11 | 25 | 4.5 |
| | | −11 | 0 | 0.5 |
| | | 11 | 25 | 4 |
| | | −11 | 0 | 0.5 |
| E/Changement | | | | |
| de tampon et fin | | | | |
| de concentration | | | | |
| [symbol] sept Sars | | | | |
| Tampon | tampon | 7 | 30 | [symbol] |
| d'analyse | d'anslyse | | | |
| F/S [symbol] peration | | | | |
| Tampon | Tampon | 7 | de 30 [symbol] 10 | 0.4 |
| d'analyse | d'analyse | | | |
| | | 7 | de 10 [symbol] 5 | 0.4 |
| | | 7 | de 5 [symbol] 0.8 | 3.2 |
| | | 7 | de 0.6 [symbol] 0.1 | 11 |
| | | 7 | de 0.1 [symbol] 0 | 0.1 |
| | | 7 | 0 | 1.9 |

[symbol]

indicates data missing or illegible when filed

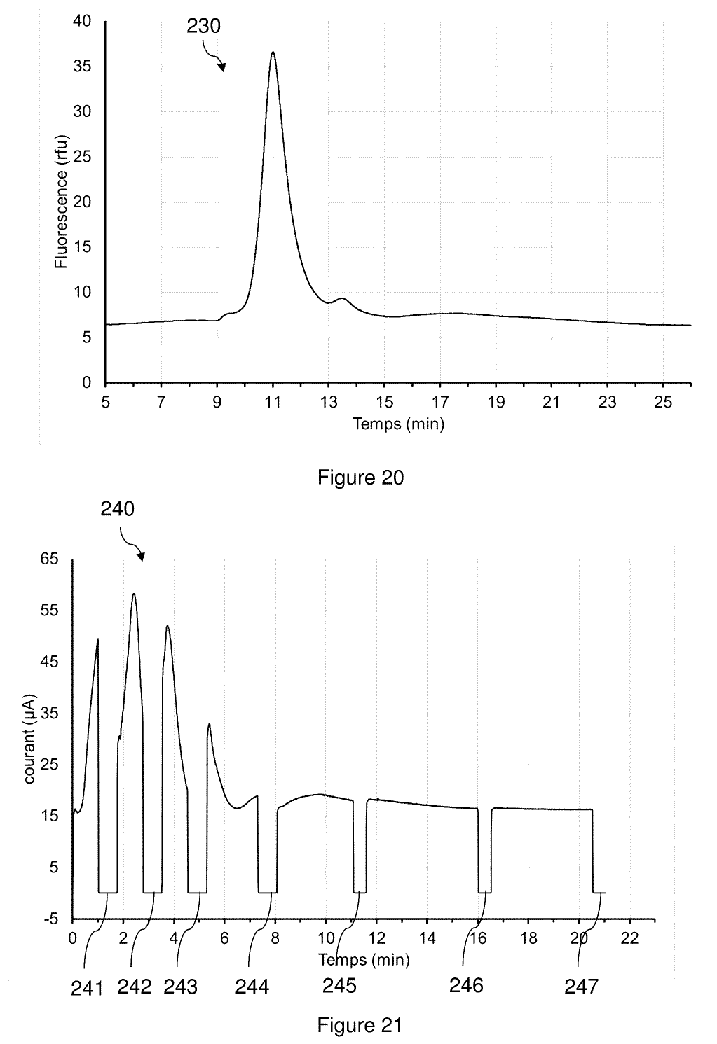

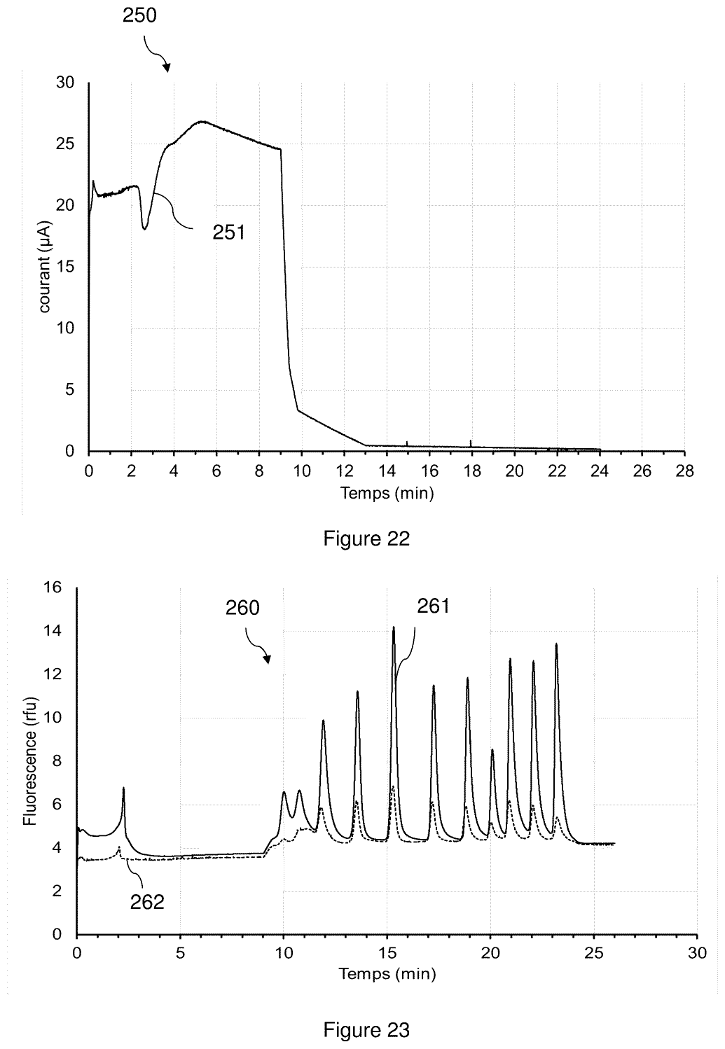

FIG. 20 shows the analysis 230 of a plasma with the seven returns method. FIG. 21 shows the current 240 obtained during the step of concentration at pH 8. This method comprises seven returns, 241 to 247. FIG. 22 shows the electrical current obtained during the last concentration step 250 with change of buffer. At about four minutes, the transition 251 between the two buffers in the separation capillary 45 are visible in the current.

The size profile in FIG. 20 is a typical profile of circulating DNA, with a main peak of approximately 165 bp, and a second, smaller, peak of approximately 305 bp.

### Example 6: Calibration of the Circulating DNA Directly in the Plasma. Annulling the Matrix Effect

The equipment and preparations of the reagents are identical to those of example 5.

The calibration samples and analysis methods are described below. In this example, the following analyses are carried out:
a pure calibration sample, analysed with the “0 returns” method, experiment identical to that of example 1; and
the same calibration sample added to a plasma sample, and analysed with the seven returns method, the same method as in example 5.

In FIG. 23, the upper curve 261 corresponds to the pure calibration sample, with 32 pg/μl, and with a zero returns method; and the lower curve 262 corresponds to the calibration sample spiked (or “added”) to the plasma, at a concentration of 9.1 pg/μl. It can be seen that the peaks of the two samples are superimposed correctly; with simply a difference in the fluorescence intensity, which corresponds to the difference in the concentration of the calibration DNA in the two analyses.

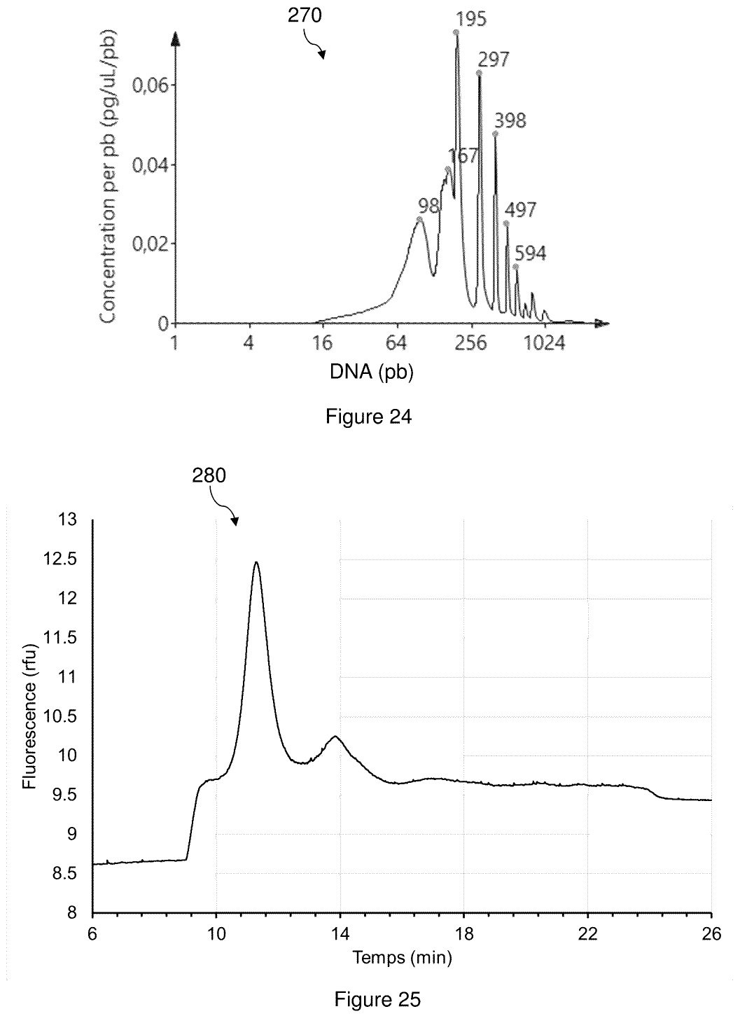

To be more precise, FIG. 24 shows the concentration curve 270 according to the size of the DNA fragments of the calibration sample added to a plasma sample, using for calibration the migration of the pure calibration sample; in other words, the curve 262 is interpreted by using the curve 261 for calibrating the size of the DNA peaks.

FIG. 24 and the table below show that the sizes of the DNA fragments of the calibration sample added to a plasma sample are determined correctly, except for the 150 bp fragment, because of the presence of the main peak of circulating DNA, which is 167 bp and which interferes with the calibration peak of 150 bp.

TABLE 8

| | Taille mesur [symbol] e quand | |
|---|---|---|
| Taille attendue des | [symbol] echantillon | |
| fragments d'ADN de | [symbol] talon est ajout [symbol] [symbol] | |
| [symbol] echantillon | un [symbol] chantillon de | Erreur |
| etalon (bp) | plasma (FIG. 24) (bp) | (en %) |
| 100 | 98 | 2% |
| 150 | 167 | |
| 200 | 195 | 2.5% |
| 300 | 297 | 1% |
| 400 | 398 | 0.5% |
| 500 | 497 | 0.6% |
| 600 | 594 | 1% |

[symbol]

indicates data missing or illegible when filed

### Example 7: Comparison of Size Profiles of Circulating DNA with or without Prior Purification of the DNA

The following equipment and methods are utilised:

A sample of plasma from a healthy donor was analysed for its circulating DNA in two ways:
Either with purification of the DNA, then analysis using the two returns method (as in example 3). The DNA was purified by means of an IDEAL-32 (registered trademark) programmable logic controller using IDXtract-MAG (registered trademark) kits from ID-Solutions (registered trademark).
Or with pre-treatment and then direct analysis without purification using the seven returns method (as in example 5).

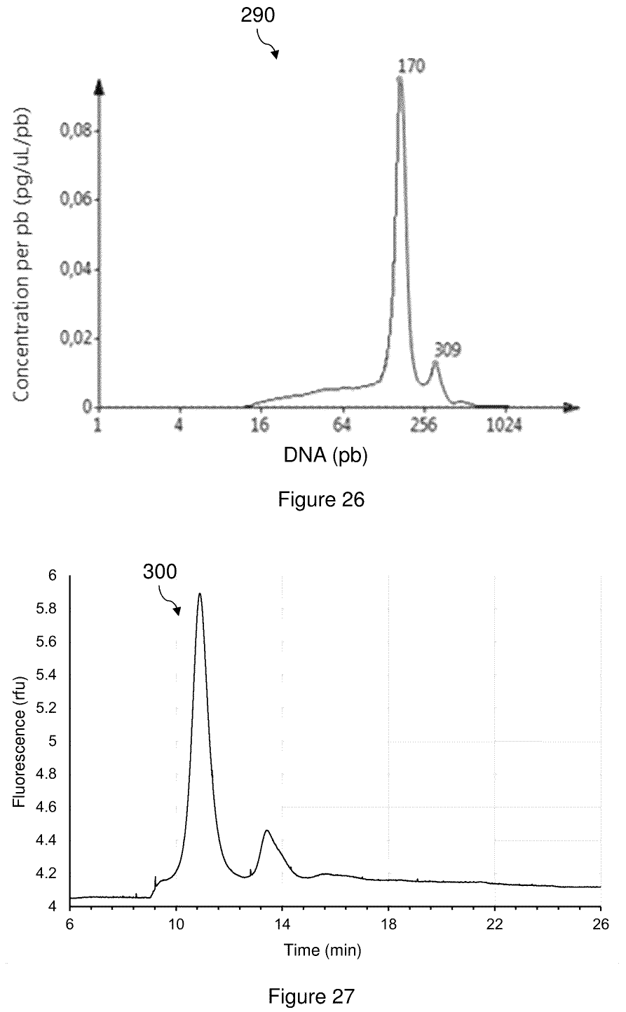

The results of the direct analysis of plasma are given, in FIG. 25, by the curve 280 of fluorescence and, in FIG. 26, by the concentration profile 290 according to the size of the DNA.

The concentration of circulating DNA in the plasma between 75 and 1650 bp was measured at 7.1 pg/μL by the seven returns method.

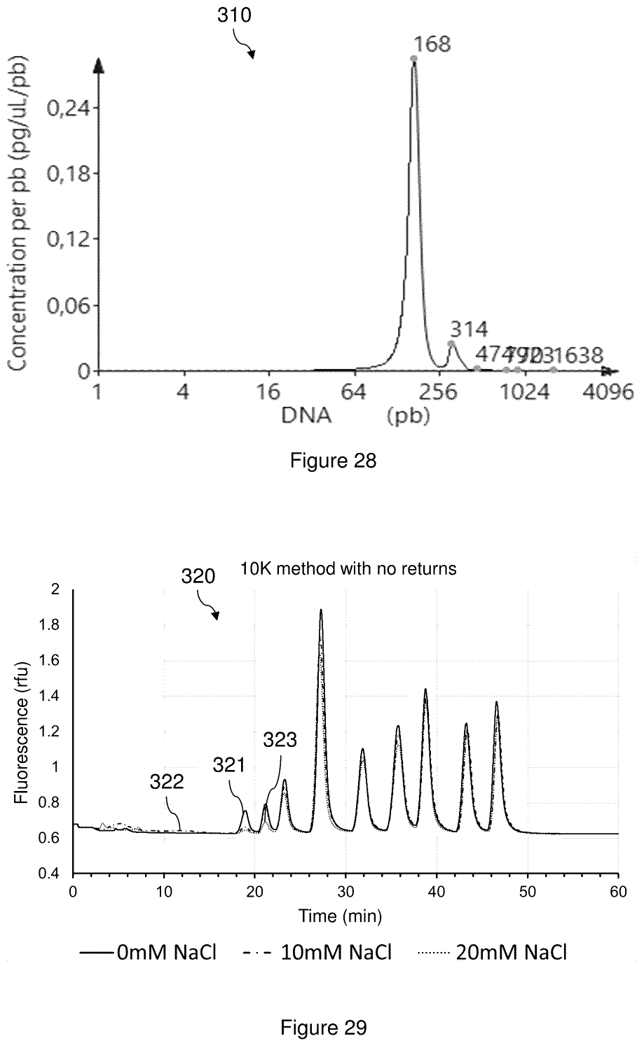

The results of the analysis with purification of the DNA are given, in FIG. 27, by the curve 300 of fluorescence, and in FIG. 28, by the concentration profile 310 according to the size of the DNA. The concentration of circulating DNA in the plasma between 75 and 1650 bp was measured at 6.7 pg/μL by the two returns method.

It can be seen that:
The concentrations of circulating DNA measured by the two methods are very close;
The sizes of the main peaks and secondary peaks are also close;
The size profile is visually close; in particular, the dissymmetry of the secondary peak, and the ratio of the secondary peak and the main peak are similar in the two methods. The seven returns method therefore makes it possible to determine the size of the circulating DNA from just a few microlitres of plasma, and without having to purify the DNA beforehand.

### Example 8: One Return Method for Large DNAs

The following equipment and methods are utilised:
Agilent CE G1600 (registered trademark);
Picometrics detector (registered trademark), Zetalif LED480 (registered trademark). (PMT set to 630V, RT set to 0.5s).

TABLE 9
Produits chimiques

| D [symbol] signation | R [symbol] f [symbol] rence | Fournisseur |
|---|---|---|
| Eau | BP2819-1 | FISHER BioReagents |
| NaOH | 28244295 | ProLabo |
| HCl | 317.1000 | MERCK |
| Tampon d'analyse | 16-BB-DNA10K/10 | Adelis Technologies |
| DNA10K | | |
| Solution de | 16-BB-DNA/09 | Adelis Technologies |
| conditionnement | | |
| Echantillon [symbol] talon | 16-BB-DNA10K/08 | Adelis Technologies |
| 10K | | |

[symbol]

indicates data missing or illegible when filed

The calibration 10K sample contains DNA fragments with the following sizes: 1 kbp, 2 kbp, 3 kbp, 4 kbp, 5 kbp, 6 kbp, 8 kbp, 10 kbp, and 20 kbp. 1 kbp equals 1000 bp, 1000 base pairs.

The concentration/separation method with no returns is described, numerically, below:

TABLE 10

| | | Pression | Voltage | Dur [symbol] e |
|---|---|---|---|---|
| Inlet | Outlet | (mBar) | (kV) | (min) |
| A/Pr [symbol] - | | | | |
| conditionnement | | | | |
| HCl 0.1M | Poubelle | 1 000 | 0 | 3 |
| PVA 1% | Poubelle | 1 000 | 0 | 3 |
| Tampon | Poubelle | 1 000 | 0 | 3 |
| B/Injection | | | | |
| Echantillon | Poubelle | 100 | 0 | 3.67 |
| Transfert | | | | |
| Tampon | Poubelle | 100 | 0 | 0.33 |
| C/Concentration | | | | |
| Tampon | Tampon | 100 | 4.5 | 15 |
| D/S [symbol] paration | | | | |
| Tampon | Tampon | 100 | de 4.5 [symbol] 1.4 | 4 |
| d'analyse | d'analyse | | | |
| | | 100 | de 1.4 [symbol] 0.9 | 5 |
| | | 100 | de 0.9 [symbol] 0.64 | 11 |
| | | 75 | 0.64 [symbol] 0.40 | 10 |
| | | 60 | 0.4 [symbol] 0.3 | 4 |
| | | 60 | 0.3 | 8 |
| | | 50 | 0.3 | 2 |
| | | 50 | de 0.3 [symbol] 0.17 | 6 |
| | | 40 | de 0.17 [symbol] 0 | 8 |
| | | 40 | 0 | 3 |

[symbol]

indicates data missing or illegible when filed

The concentration/separation method on large DNAs with one return is described, numerically, below:

TABLE 11

| | | Pression | Voltage | |
|---|---|---|---|---|
| Intet | Outlet | (mBar) | (kV) | Dur [symbol] e (s) |
| A/Pr [symbol] - | | | | |
| conditionnement | | | | |
| HCl 0.1M | Poubelle | 1 000 | 0 | 2 |
| PVA 1% | Poubelle | 1 000 | 0 | 3 |
| Tampon | Poubelle | 1 000 | 0 | 3 |
| B/Injection | | | | |
| Echantillon | Poubelle | 100 | 0 | 3.33 |
| B/Transfert | | | | |
| Tampon | Poubelle | 100 | 0 | 0.5 |
| d'analyse | | | | |
| C/Concentration | | | | |
| Tampon | Tampon | 100 | 4.5 | 8 |
| d'analyse | d'analyse | | | |
| | | −100 | 0 | 1 |
| | | 100 | 4.5 | 5 |
| D/S [symbol] paration | | | | |
| Tampon | Tampon | 100 | de 4.5 [symbol] 1 | 5 |
| d'analyse | d'analyse | | | |
| | | 80 | de 1 [symbol] 0.3 | 23 |
| | | 80 | de 0.3 [symbol] 0 | 2 |
| | | 80 | 0 | 2 |

[symbol]

indicates data missing or illegible when filed

To evaluate the effect of the presence of salts in the concentration of a sample of large-sized DNA, NaCl was added to the calibration 10K sample, respectively at concentrations of 0, 10 and 20 mM.

The results of the analyses 320 of the calibration DNA samples, for the DNA 10K method with no returns, are shown in FIG. 29; 0 mM NaCl by curve 321, 10 mM NaCl by curve 322, and 20 mM NaCl by curve 323.

It can be seen that, when 10 mM NaCl is added to the calibration sample, the 1 kb fragment is not retained correctly. With 20 mM NaCl in the sample, the 1 kbp fragment has almost disappeared, and the 2 kbp fragment is no longer retained correctly either.

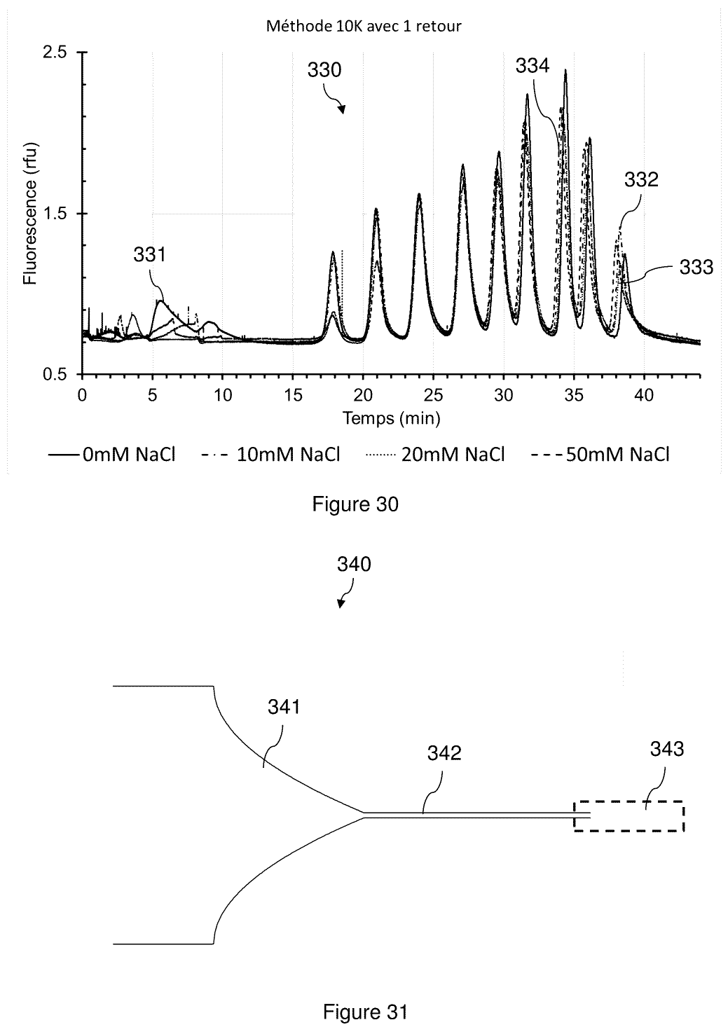

The results of the analyses 330, with the addition of a return between concentration minute eight and minute nine, and some modifications in the time and gradient to accelerate the method, are shown in FIGS. 30:
0 mM NaCl by curve 331;
with 10 mM NaCl by curve 332;
with 20 mM NaCl by curve 333; and
with 50 mM NaCl by curve 334.

By introducing a return between minute eight and minute nine, the 1 kbp fragment is retained correctly during the concentration phase. At 20 mM NaCl, the 1 kbp fragment is not completely retained, but the 2 kbp fragment is now completely retained.

### Example 9: “Microfluidic Chip” Type of Device

Examples 1 to 8 utilise capillary devices 40, shown schematically in FIG. 1. The method that is the subject of the present invention also applies to microfluidic channels. The present invention can therefore be implemented in microfluidic chips having a geometric shape configured so that the small DNA fragments leak slowly if the sample has high conductivity. For example, in the shape shown in FIG. 31, the flow area, i.e. the injection chamber 341 and the capillary 342 corresponding to the narrow channel, have a depth (measured perpendicularly in FIG. 31) of two micrometres. The image capture area 343 is partially shown on the right in FIG. 31. In this image capture area, the narrow channel 342 downstream from the constriction has a width, measured from top to bottom in FIG. 31, of 15 μm.

It is noted that the depth, here 2 μm, can be increased, for example, up to 50 μm, and that the width, here 15 μm, can vary, for example, between 2 and 50 μm.

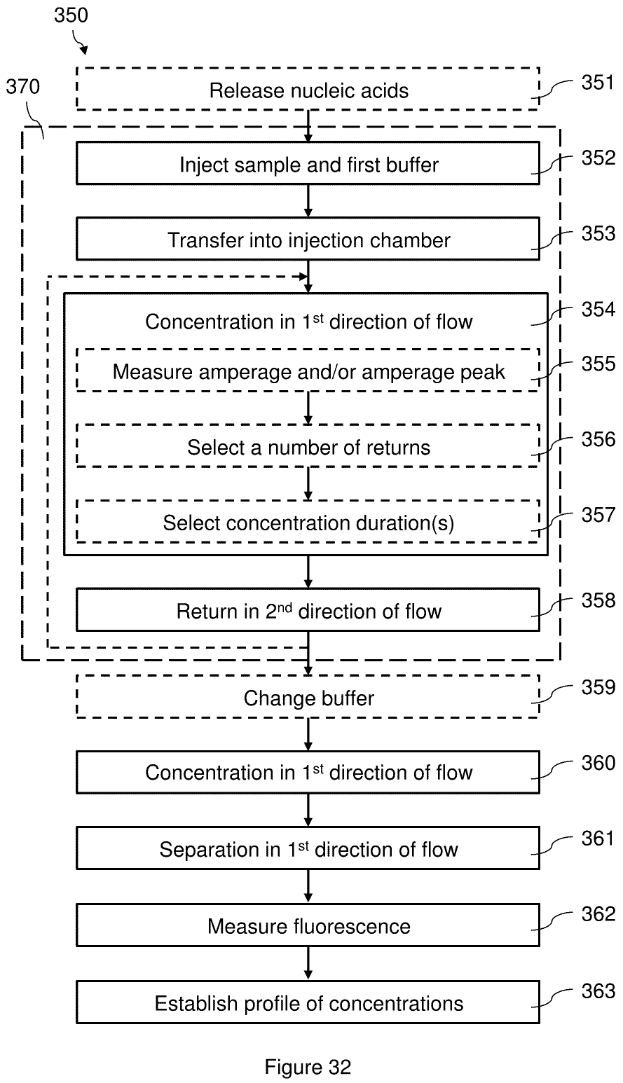

FIG. 32 shows, in the form of a logical diagram, steps in a particular embodiment of the desalting and concentration method 370 that is the subject of the invention and the analysis method 350 that is the subject of the invention.

The method 370 for desalting and concentrating a nucleic acid sample more conductive than an analysis buffer, comprises firstly a step 352 of injecting the sample comprised of nucleic acid and the first buffer into the device. During a step 353, the sample is transferred into the injection chamber 44. During a step 354, a laminar flow of the sample into a capillary equipped with a local restriction in its cross-section, in a first direction of flow, is carried out. During this flow, the sample is subjected to a first difference in electric potential whose action on the nucleic acid molecules is opposite to the first direction of flow and causes the nucleic acid molecules to be retained in the capillary, or at least slowed down by pLAS effect in the separation capillary 45.

During an optional step 355, the amperage circulating in the capillary during the step 354 is measured. During an optional step 356, a number of iterations of an alternation of steps 354 and 358 is selected according to the amperage measured. Preferably, the selected number of iterations of the alternation is an increasing function of the amperage of a peak of this measured amperage or of a peak of a derivative of this measured amperage.

When the number of returns is not determined by a measurement of the salinity of the sample, it is predefined, for example with a value given in one of examples 3 to 8.

For example, the number of returns, and therefore of iterations, is predefined according to the minimum size of nucleic acids to be concentrated. Thus, a favourable salinity is defined based on a minimum threshold set by the size of the nucleic acids to be concentrated. During the implementation of the method that is the subject of the invention, it is noted that the sizes of the nucleic acids retained are, in particular, dependent on the flow speed, the voltage applied and the nature of the analysis buffer such as its conductivity.

During an optional step 357, a duration of at least one step 354 of flowing in the first direction of flow is selected. Preferably, the duration chosen is a decreasing function of the amperage of a peak of this measured amperage or of a peak of a derivative of this measured amperage.

When the duration of at least one concentration step is not determined by a measurement of the salinity of the sample, it is predefined, for example with a value given in one of examples 3 to 8.

At the end of the selected or predefined duration of step 354, during a return step 358, a laminar flow of the sample, in a second direction of flow opposite to the first direction of flow, is carried out. In some preferred embodiments, during the step 358 of the laminar flow of the sample in the second direction of flow, the sample is subjected to a second difference in electric potential whose action on the nucleic acid molecules is opposite to the first direction of flow.

In the case where the selected or predefined number of iterations of the alternation of steps 354 and 358 is greater than one then, at the end of step 358, one returns to step 354. Note that, in the following iterations, the optional steps 355 to 357 can be omitted.

At the end of all the iterations of steps 354 to 358, the method for desalting and concentrating nucleic acids is finished and the sample is removed from the device.

In the method 350 for analysing nucleic acids, an optional step 351 of releasing nucleic acids, with or without purification, can precede the iterations of steps 354 to 358.

If the release of nucleic acids leaves proteins in the sample then, following the last iteration of steps 354 to 358, during an optional step 359, a change of analysis buffer is carried out, the new analysis buffer having a lower pH than the analysis buffer previously used. This is, for example, the case of the release of nucleic acids, such as the proteinase K digestion in example 5. In this case, the sample is a biological sample and the change of buffer/pH of step 359 is preferably before the steps 360 to 363 are carried out. If there are no proteins in the sample, the steps 352 to 358 are carried out with an analysis buffer with a pH close to neutral, or slightly acidic, and, after the last iteration of steps 354 to 358, one proceeds directly to a step 360.

During this step 360, a laminar flow of the sample into a capillary equipped with a local restriction in its cross-section, in the first direction of flow, is carried out. During this flow, the sample is subjected to a difference in electric potential whose action on the nucleic acid molecules is opposite to the first direction of flow and causes the nucleic acid molecules to be retained in the capillary.

During a step 361, a separation by laminar flow of the sample in the capillary is carried out in the first direction of flow. Preferably, during this flow, the sample is subjected to a difference in electric potential less than or equal to the first difference in potential, whose action on the nucleic acid molecules is opposite to the first direction of flow and causes a partial retention of the nucleic acid molecules in the capillary. Preferably, the difference in electric potential decreases during the step 361, either in steps or decreasing continually, i.e. with a negative, non-zero time derivative.

During a step 362, carried out during or after the separation step, a measurement is made of a fluorescence time profile of fluorescent nucleic acid molecules and a step is carried out of converting the fluorescence time profile into a concentration profile of nucleic acid molecules of different lengths. Preferably, for this conversion step, the fluorescence profile of a calibration sample, the concentration of which is known for each molecule length of nucleic acids, is used to offset experimental variations that exist from one day to the next, in terms of the passage time of the DNA in front of the detector and in terms of fluorescence intensity.

1. A method for desalting and concentrating a nucleic acid sample more conductive than an analysis buffer, comprising at least one iteration of an alternation of:
a step of the laminar flow of the sample through a capillary in a first direction of flow, the capillary being equipped with at least one local restriction in its cross-section and comprising the analysis buffer, during which the sample is subjected to a first difference in electric potential whose action on the nucleic acid molecules is opposite to the first direction of flow and causes the nucleic acid molecules to be retained in the capillary;
a step of the laminar flow of the sample, in a second direction of flow opposite to the first direction of flow.

2. The method according to claim 1, wherein, during the step of the laminar flow of the sample in the second direction of flow, the sample is subjected to a second difference in electric potential whose action on the nucleic acid molecules is opposite to the first direction of flow.

3. The method according to claim 1, which also comprises a step of measuring the amperage circulating in the capillary during at least one step of flowing in the first direction of flow and a step of selecting a number of iterations of the alternation according to the amperage measured.

4. The method according to claim 3, wherein the number of iterations of the alternation is an increasing function of the amperage of a peak of this measured amperage or of a peak of a derivative of this measured amperage.

5. The method according to claim 1, which also comprises a step of measuring the amperage circulating in the capillary during at least one step of flowing in the first direction of flow and a step of selecting a duration of at least one step of flowing in the first direction of flow according to the amperage measured.

6. The method according to claim 5, wherein the duration chosen is a decreasing function of the amperage of a peak of this measured amperage or of a peak of a derivative of this measured amperage.

7. The method according to claim 1, which comprises after an iteration of the alternation, a step of changing the analysis buffer, the new analysis buffer having a lower pH than the analysis buffer previously used.

8. The method for analysing a nucleic acid sample, which comprises the steps of the method for desalting and concentrating a nucleic acid sample according to claim 1, and, after the last iteration of the alternation, a step of separation by laminar flow of the sample in the capillary in the first direction of flow, during which the sample is subjected to a difference in electric potential less than or equal to the first difference in potential, whose action on the nucleic acid molecules is opposite to the first direction of flow and causes a partial retention of the nucleic acid molecules in the capillary.

9. The method according to claim 8, wherein, during the separation step the difference in electric potential decreases.

10. The method according to claim 8, which comprises, during or after the separation step, a step of measuring a fluorescence time profile of fluorescent nucleic acid molecules and a step of concentrating into nucleic acid molecules of different lengths, by utilising a fluorescence profile of a calibration sample, in which the concentration is known for each length of fluorescent nucleic acid molecule.

11. A device configured to implement a desalting and concentration method according to claim 1, or an analysis method for analysing a nucleic acid sample more conductive than an analysis buffer, the device comprising:
a capillary equipped with a local restriction in its cross-section and comprising the analysis buffer;
a means for placing the sample in the capillary in laminar flow, in a first direction of flow;
a means for applying a first difference in electric potential during the flow in the first direction, a difference in potential whose action on the nucleic acid molecules is opposite to the first direction of flow and causes the retention of the nucleic acid molecules in the capillary;
a means for placing the sample in the capillary in laminar flow, in a second direction of flow opposite to the first direction of flow; and
a means for controlling the means for placing in laminar flow and the means for applying the first difference in electric potential configured to control at least one iteration of one alternation;
a laminar flow of the sample in the capillary in the first direction of flow, a flow during which the sample is subjected to the first difference in electric potential; and
a laminar flow of the sample in the capillary in the second direction of flow.

12. The device according to claim 11, wherein the capillary is formed of a microfluidic channel.
